(19) United States
(12) Patent Application Publication
(10) Pub. No.: US 2015/0045407 A1
(43) Pub. Date: Feb. 12, 2015

(54) MTK1-ACTIN INHIBITORS AND METHODS OF USE

(71) Applicants: Richard R. Vaillancourt, Tucson, AZ (US); James J. Sollome, Tucson, AZ (US)

(72) Inventors: Richard R. Vaillancourt, Tucson, AZ (US); James J. Sollome, Tucson, AZ (US)

(73) Assignee: THE ARIZONA BOARD OF REGENTS ON BEHALF OF THE UNIVERSITY OF ARIZONA, Tucson, AZ (US)

(21) Appl. No.: 14/453,497

(22) Filed: Aug. 6, 2014

Related U.S. Application Data
(60) Provisional application No. 61/862,888, filed on Aug. 6, 2013.

(51) Int. Cl. A61K 31/403; G01N 33/50

(52) U.S. Cl. CPC A61K 31/403; G01N 33/5011; G01N 2500/02
USPC 514/411; 435/7.9

(57) ABSTRACT

One aspect of the invention provides a method for treating a clinical condition associated with inappropriate or abnormal activation of HER3/ErbB3. In some embodiments, the method of the invention inhibits interaction between MTK1 and actin.

### CROSS-REFERENCE TO RELATED APPLICATIONS

This application claims the priority benefit of U.S. Provisional Application No. 61/862,888, filed Aug. 6, 2013, which is incorporated herein by reference in its entirety.

### STATEMENT REGARDING FEDERALLY FUNDED RESEARCH

This invention was made with government support under Grant Nos. RO1 ES012007 and T32 ES007091 awarded by the National Institutes of Health (NIH). The government has certain rights in the invention.

### FIELD OF THE INVENTION

The present invention relates to modulating interaction between MTK1 and actin and methods for using the same for various applications. In one particular embodiment, modulation of such interaction results in modulation of activation of HER3/ErbB3 by the hormone heregulin. In some instances, modulation of interaction between MTK1 and actin is used to treat a various clinical conditions.

### BACKGROUND OF THE INVENTION

Receptor tyrosine-protein kinase erbB-3, also known as HER3 (human epidermal growth factor receptor 3), is a membrane bound protein. In humans, HER3/ErbB3 is encoded by the ERBB3 gene. HER3/ErbB3 is a member of the epidermal growth factor receptor (EGFR/ERBB) family of receptor tyrosine kinases. HER3/ErbB3 has been shown to bind to inter alia heregulin. It has also been shown that binding of heregulin to HER3/ErbB3 causes a change in conformation that allows for dimerization, phosphorylation, and activation of signal transduction.

It has been shown that during human development, ERBB3 is expressed in skin, bone, muscle, nervous system, heart, lungs, and intestinal epithelium. ERBB3 is believed to be also expressed in normal adult human gastrointestinal tract, reproductive system, skin, nervous system, urinary tract, and endocrine system. Inappropriate activation of HER3/ErbB3 is believed to contribute to the etiology and progression of any of the various clinical conditions associated with cells that express ERBB3 including various cancers. However, none of the conventional clinical treatment methods target modulating HER3/ErbB3 activation.

Accordingly, there is a need for a method for modulating HER3/ErbB3 for various clinically related applications.

### SUMMARY OF THE INVENTION

Some aspects of the invention provide a method for modulating activation of HER3/ErbB3. In one particular aspect of the invention, modulation of HER3/ErbB3 activation is achieved by inhibiting interaction between MTK1 and actin. Yet another aspect of the invention provides a method for modulating interaction between MTK1 and actin. The term “modulate” refers to both activation or increase in as well as inhibition or decrease in the activity and/or the expression or transcription of the corresponding genes and/or peptides. For treating a clinical condition associated with HER3/ErbB3, the method of invention inhibits activation of HER3/ErbB3. The term “inhibit’ refers to decrease, reduce, or prevent activation of HER3/ErbB3. Such inhibition can be achieved by inhibiting the activity of HER3/ErbB3 itself, inhibiting the translation and/or transcription of ERBB3 gene and/or the corresponding mRNA.

Another aspect of the invention provides a method for treating a clinical condition associated with activation of HER3/ErbB3. Such a method typically includes optionally identifying a subject in need of a treatment associated with activation of HER3/ErbB3; and administering to said subject a therapeutically effective amount of a mitogen-activated protein three kinase (MTK1)-actin inhibitor. It should be appreciated that methods of the invention are directed to treating a clinical condition associated with activation, i.e., abnormal or unregulated activation, of HER3/ErbB3. Such a clinical condition is detrimental or harmful to the health of the subject.

In one particular embodiment, said clinical condition associated with activation of HER3/ErbB3 comprises cancer, lung fibrosis, chronic obstructive pulmonary disease, or acidification of tumor metabolic microenvironment.

Yet in other embodiments, said clinical condition associated with activation of HER3/ErbB3 comprises breast cancer, prostate cancer, ovarian cancer, neurological cancers, retinoblastomas, melanoma, gastrointestinal cancers, pancreatic cancer, colorectal cancers, head and neck cancers, or lung cancer.

Still in other embodiments, said MTK1-actin inhibitor comprises cytochalasin D.

In other embodiments, siRNA is used to decrease the amount of MTK1 expression, which effectively reduces the amount of MTK1 available for MTK1-actin interaction. Suitable siRNAs that can inhibit MTK1 expression can be readily determined by one skilled in the art as the gene sequence for MTK1 is known.

Still in other embodiments, said MTK1-actin inhibitor comprises a molecule that can interact with or bind to (and thus inhibit interaction between MTK1 and actin) any of the amino acid sequences 264-303 of MTK1.

Still another aspect of the invention provides a method for screening for or identifying an anticancer compound. Such a method can be used as a research tool and/or to determine the lead compound for further research. Typically, such a method comprises: contacting a cell culture with a test compound under conditions sufficient to determine whether the test compound modulates interaction between actin and a mitogen-activated protein kinase kinase (MTK1); and selecting the test compound that inhibits interaction between actin and MTK1 as an anticancer compound.

In some embodiments, said cell culture comprises cancerous cells. In one particular embodiment, said cell culture comprises HEK-293, T-47D, MCF7, or MDA-MB-231 cells or a combination thereof.

Another aspect of the invention provides a method for treating a patient suffering from a cancer. Such a method typically comprises administering to the patient in need of such a treatment a therapeutically effective amount of a composition comprising a molecule that inhibits interaction between actin and mitogen-activated protein three kinase (MTK1). Alternatively, the method can include administering to the patient in need of such a treatment a therapeutically effective amount of a composition comprising a molecule that inhibits activation of HER3/ErbB3. Type of cancers that can be treated by the method of invention include, but are not limited to, breast cancer, prostate cancer, ovarian cancer, neurological cancers, retinoblastomas, melanoma, gastrointestinal cancers, pancreatic cancer, colorectal cancers, head and neck cancers, and lung cancer.

### BRIEF DESCRIPTION OF THE DRAWINGS

FIG. 1 shows that HER3 associates with MTK1 in response to HRG stimulation. A schematic representation of the proline-rich region (PPP), actin-interacting region (AIR), alanine-rich region (AAA), and catalytic domain of MTK1 is shown. The MTK1 antibody recognizes an amino terminal region (amino acids 18-139) of MTK1 (Panel A). T-47D cells were stimulated with 10 nM HRG for 12 minutes or 0.3 M sorbitol for 30 minutes as indicated (Panel B). MTK1 was immunoprecipitated and proteins were resolved by SDS-PAGE and immunoblotted using monoclonal antibody 4G10 (pTyr). T-47D cells were stimulated with HRG and MTK1 was immunoprecipitated as described above (Panels C & D). Immunoblots were performed using antibodies directed against EGFR and HER2 (Panel C) or HER3 and HER4 (Panel D). Membranes were stripped and immunoblotted with MTK1 antibody (B, C and D, bottom panels).

FIG. 2 shows that HER3 associates with MTK1 in both T-47D and MCF-7 cells. Cells were stimulated as described in FIG. 1. Cell lysates were incubated with antibody directed against phosphotyrosine (pTyr), MTK1, or normal rabbit IgG. Proteins were resolved by SDS-PAGE and immunoblotted with anti-phosphotyrosine antibody (A, top panel) or antibody specific for phosphotyrosine 1289 of HER3 (B, top panel). The membranes were re-probed for MTK1 (bottom panels). MCF-7 cell lysates were prepared as described above and a fraction of the immunoprecipitate (15%) was immunoblotted using anti-phosphotyrosine antibody (Panel C, lanes a & b), while the remaining immunoprecipitation was silver stained (lanes c & d). HER3 peptides were identified in the HRG stimulated sample (arrows) by LC-MS/MS with 99.9% confidence (data not shown).

FIG. 3 shows sustained MTK1 and HER3 association. MCF-7 cells were stimulated with 10 nM HRG for the indicated times. MTK1 was immunoprecipitated and proteins were resolved by SDS-PAGE, then immunoblotted with anti-phosphotyrosine antibody (A, top panel). The membrane was re-robed with MTK1 antibody (bottom panel). T-47D cells were stimulated with 10 nM HRG for 12 minutes, 3.3 nM EGF for 12 minutes, 10 μg/ml insulin for 15 minutes or 0.3 M sorbitol for 30 minutes followed by immunoprecipitation of MTK1. The proteins were resolved by SDS-PAGE and immunoblotted with the indicated antibodies.

FIG. 4 shows that HER2 kinase activity was required for MTK1/HER3 association. MCF-7 cells were pre-treated with 0, 50, 100 or 200 nM lapatinib for 1 hour followed by 10 nM HRG stimulation for 12 minutes. Phosphotyrosine immunoblot analysis of MTK1 immunoprecipitations show 50 nM lapatinib attenuates MTK1/HER3 association (Panel A, lane c). Cell extracts (150 μg) that were used for the immunoprecipitation were resolved by SDS-PAGE and immunoblotted for phosphotyrosine (B, top panel). HER2 negative, but HER3 positive MDA-MB-231 cells were stimulated with 10 nM HRG followed by MTK1 immunoprecipitations and compared to T-47D and MCF-7 cells. Phosphotyrosine immunoblot analysis shows HER2 is required for MTK1/HER3 association in response to HRG (Panel C, lane c). Membranes were stripped and immunoblotted for MTK1 (A, B and C, bottom panels).

FIG. 5 shows that actin associates with MTK1 and inhibition of actin polymerization with cytochalasin D inhibits MTK1/HER3 association. Comparison of mouse MTK1 and the actin interacting region (AIR) of the yeast homolog Ssk2p showed 17/40 amino acids were identical and 25/40 were conserved. Amino acids 256-295 were deleted from the mouse MTK1 cDNA (AIR mutant). HEK-293 cells were transfected with empty vector, flag-tagged wild-type mouse MTK1 or the flag-tagged AIR mutant mouse MTK1 cDNA. After 48 hours, the cells were stimulated with 20% fetal bovine serum or 0.3M sorbitol. Flag antibody-beads were used for immunoprecipitation for flag-tagged proteins. Proteins were resolved by SDS-PAGE and immunoblotted with antibody that recognizes the FLAG epitope (B, top panel) or actin (lower panel). Cell lysates were resolved by SDS-PAGE and immunoblotted with the indicated antibodies. MCF-7 cells were pre-treated with 1 μg/ml cytochalasin D (Cyto D) for 30 minutes followed by 10 nM HRG for 12 minutes or 0.3M sorbitol for 30 minutes. MTK1 was then immunoprecipitated from each condition (C, lanes a-f). Antibody directed against phosphotyrosine 1289 of HER3 was used to immunoblot (top panel). The membrane was re-probed for MTK1 (bottom panel). Cell lysates were resolved by SDS-PAGE and immunoblotted as indicated. Cytochalasin D had no effect on the phosphorylation of HER3 (lanes g & j), but diminished interaction between MTK1 and HER3 (lanes a & d).

FIG. 6 shows that MTK1 is required for HRG induced extracellular acidification. MCF-7 cells were stimulated with 10 nM HRG or 3.3 nM EGF, after 24 hours the cells were counted (Panel A, left graph) and the pH of the media was measured (right graph). MTK1 siRNA knockdown was used to determine whether MTK1 is required for HRG induced extracellular acidification. MCF-7 cells were transfected as described in the methods section. The cells were then stimulated with 10 nM HRG for 24 hours followed by pH measurements of the media (Panel B). Non-transfected or cells pre-treated with 250 nM lapatinib were also stimulated with HRG followed by pH measurements (Panel B). MTK1 knockdown did not have an effect on cell proliferation compared to NS knockdown (Panel C). Statistical analysis was performed using a two-sided student t-test with standard deviations and a minimum of three replicates, p-value of ≦0.05 was considered statistically significant and each experiment was repeated a minimum of three independent times.

FIG. 7 shows that MTK1 is required for HRG induced cell migration. MCF-7 cells were transfected as described previously. The cells were serum starved for four hours, then scratched followed by HRG stimulation. Digital images were acquired at 0, 24 and 48 hours post scratch (Panel A). Statistical analysis revealed MTK1 knockdown cells migrated by a mean of 17.3%±4.6 (Panel B, lane b) compared to a NS knockdown mean of 55.3%±6.4 (Panel B, lane c). Similar results were observed at 48 hours with MTK1 knockdown migrating by a mean of 40.1%±6.8 (Panel C, lane b) compared to NS knockdown by a mean of 89%±7.8 (Panel C, lane c). Statistical analysis was performed as described in FIG. 6. To measure knockdown efficiency of MTK1, cell extracts (150 μg) were resolved by SDS-PAGE, followed by MTK1 immunoblot analysis and densitometry measurements using BioRad Image Lab software to detect extent of MTK1 knockdown (Panel D, lane b), actin was used as a loading control (bottom panel).

### DETAILED DESCRIPTION OF THE INVENTION

HER2 (Human Epidermal Growth Factor Receptor 2) also known as Neu, ErbB-2, CD340 (cluster of differentiation 340) or p185 is a protein that is encoded by the ERBB2 gene. HER2 is also a member of the epidermal growth factor receptor (EGFR/ErbB) family. Inappropriate activation of the HER2/ErbB2-HER3/ErbB3 receptor heterodimer in breast cancer, as well as other cancers, results in unregulated cell growth and poor patient prognosis. Amplification or over-expression of ERBB2 gene has been shown to play an important role in the pathogenesis and progression of certain aggressive types of breast cancer and in recent years it has evolved to become an important biomarker and target of therapy for the disease. Currently, the catalytic activity and over-expression of HER2/ErbB2 are clinically typically treated with lapatinib and trastuzumab, respectively.

Human MAP3K4 (MTK1) functions upstream of mitogen activated protein kinases (MAPKs). The present inventors have discovered that MTK1 is required for human epidermal growth factor receptor 2/3 (HER2/HER3)-heregulin β1 (HRG) induced cell migration in MCF-7 breast cancer cells. The present inventors have also discovered that HRG stimulation leads to association of MTK1 with activated HER3 in MCF-7 and T-47D breast cancer cells. Without being bound by any theory, it is believed that activated HER3 association with MTK1 is dependent on HER2 activation and is decreased by pre-treatment with the HER2 inhibitor, lapatinib.

Some aspects of the present invention are based on the discovery by the present inventors of a signaling pathway involving activated HER3/ErbB3. In particular, the present inventors have discovered that activated HER3/ErbB3 physically interacts with the serine/threonine kinase, MTK1. Specifically, the present inventors have discovered that amino acids 264-303 within MTK1 are involved in the interaction between actin and MTK1. Interestingly, the present inventors have discovered that activation of the HER2/ErbB2-HER3/ErbB3 receptor heterodimer with heregulin, but in the presence of cytochalasin D which prevents actin polymerization, disrupted the communication or signaling between HER3/ErbB3 and MTK1. Surprisingly and unexpectedly, it was discovered that the activation of HER3/ErbB3 was not affected by cytochalasin D, indicating that the actin cytoskeleton is not required for HER3/ErbB3 receptor activation, but the actin cytoskeleton is required for communication or signaling between the HER3/ErbB3 receptor and intracellular proteins like MTK1.

MTK1 interacts with ERK1/2 constitutively and activation of HER3/ErbB3 results in ERK1/2 activation. ERK1/2 is known to activate other intracellular proteins needed for cellular proliferation. It is believed that inhibition of the communication between HER3/ErbB3-MTK1-ERK1/2 provides a molecular approach to prevent HER3/ErbB3-mediated cellular proliferation. One method of disrupting communication along the chain of HER3/ErbB3-MTK1-ERK1/2 is to prevent HER3/ErbB3 and MTK1 from interacting with actin.

The HER3/ErbB3 tyrosine kinase receptor lacks catalytic activity but is phosphorylated on tyrosine by other tyrosine kinases (i.e., HER2/ErbB2 & Src). These phosphorylation sites serve as recruitment sites for other proteins such as phosphatidylinositol 3-kinase (PI3K). Since the HER3/ErbB3 tyrosine kinase receptor lacks catalytic activity, it cannot be inhibited by using inhibitors of the catalytic domain. However, one way to inhibit the HER3/ErbB3 tyrosine kinase receptor is to inhibit the function of proteins that interact with the receptor.

The present inventors have discovered that the serine/threonine kinase, MTK1 (human nomenclature) or MEKK4 (mouse nomenclature), physically interacts with the HER3/ErbB3 tyrosine kinase receptor, but only in response to the growth factor, heregulin. Thus, only the activated HER3/ErbB3 receptor interacts with MTK1. The present inventors have also discovered that the actin interacting region of MTK1 (amino acids 264-303) is required for the interaction between MTK1 and HER3/ErbB3. Accordingly, some aspects of the invention are based on targeting the actin interacting region of MTK1 with molecules to inhibit the formation of the MTK1-actin complex, thereby preventing downstream signaling between MTK1 and HER3/ErbB3.

Cell proliferation occurs when the HER3/ErbB3 receptor is activated by heregulin. For example, it was discovered that knockdown of MTK1 expression resulted in loss of heregulin-dependent proliferation of the MCF7 breast cancer cell line, which indicates that MTK1 is required for heregulin dependent cell proliferation. Interrupting the interaction between MTK1 and HER3/ErbB3 with molecules that target the actin interacting region of MTK1 is believed to inhibit heregulin dependent cell proliferation in vivo. Thus, other aspects of the invention provide methods for using compounds or molecules that are useful therapeutic agents to treat patients with HER3/ErbB3-dependent cancers.

Phosphorylation of the HER3/ErbB3 tyrosine kinase receptor is highly dependent on the catalytic activity of the HER2/ErbB2 tyrosine kinase receptor. HER2/ErbB2 is often over-expressed in cancer and the tyrosine kinase activity of HER2/ErbB2 contributes to the phosphorylation of the HER3/ErbB3 receptor. Trastuzumab & pertuzumab are antibodies that bind to extracellular domain of HER2/ErbB2 and lapatinib is a small molecule that binds to the kinase catalytic domain of HER2/ErbB2. These antibodies and small molecule inhibit HER2/ErbB2 and thereby indirectly inhibit the phosphorylation of HER3/ErbB3. However, patient resistance to these drugs occurs indicating that other proteins contribute to cancer. Unlike other conventional cancer treatment methods, methods of the invention target inhibition of HER3/ErbB3-dependent activation of ERK1/2 by inhibiting the interaction between MTK1 and actin, which is required for the recruitment of MTK1 to HER3/ErbB3 in response to heregulin.

Some aspects of the invention are based on the discovery and identification by the present inventors of the actin interacting region (AIR) on MTK1. In particular, the present inventors have discovered that disruption of actin cytoskeletal polymerization with cytochalasin D inhibited HRG induced MTK1/HER3 association. Additionally, HRG stimulation led to extracellular acidification that was independent of cellular proliferation. HRG induced extracellular acidification was significantly inhibited when MTK1 was knocked down in MCF-7 cells. Similarly, pre-treatment with lapatinib significantly decreased HRG induced extracellular acidification. Extracellular acidification is linked with cancer cell migration. Scratch assays showed HRG induced cell migration in MCF-7 cells. Knockdown of MTK1 significantly inhibited HRG induced cell migration. Furthermore, pre-treatment with lapatinib also significantly decreased cell migration. Cell migration is required for cancer cell metastasis, which is the major cause of cancer patient mortality. Other aspects of the invention are based on the identification of MTK1 in the HER2/HER3-HRG mediated extracellular acidification and cell migration pathway in breast cancer cells.

Mitogen activated protein kinases (MAPKs) are regulated by various extracellular stimuli resulting from a cascade of sequential phosphorylations. MAPKs, such as the extracellular signal-regulated kinases (ERKs), are phosphorylated by MEKs and MEKs are phosphorylated by MEKKs (Lange-Carter et al., 1993). The MEKK family of MAP3Ks was cloned based on homology to the catalytic domain of the yeast MAP3K, Ste11 (Lange-Carter et al., 1993). MEKK4 (MAP3K4) was cloned using cDNA isolated from mouse (Gerwins et al., 1997), while MTK1 (MAP3K4) was cloned using human cDNA (Takekawa et al., 1997) and the sequence homology between the two proteins is 88% amino acid identity and 92% amino acid homology. When Ssk2 was cloned from yeast (Maeda et al., 1995) it became apparent that the MEKK4 and MTK1 amino acid sequences are more homologous to yeast Ssk2p than Stel1p (Bettinger and Amberg, 2007). Ssk2p is regulated by osmotic stress (Takekawa et al., 1997). In yeast lacking Ssk2p, MEKK4 rescues the loss of Ssk2p resulting in p38 MAPK activation indicating that MEKK4 compliments Sskp2 in yeast (Takekawa et al., 1997).

The heart is one of the first organs to develop and congenital malformations occur at a rate of about one in one hundred (Hoffman and Kaplan, 2002). Mutation of lysine in the active site of MEKK4 produces a kinase inactive protein. Kinase inactive MEKK4 attenuates developmental epithelial to mesenchymal transformation in mouse atrioventricular canal and ventricular heart explants (Stevens et al., 2006). When a knock-in mutation of kinase-inactive MEKK4 was introduced in mice, the pups died at birth from skeletal malformations and neural tube defects (Abell et al., 2005). These findings emphasize the importance of MEKK4 kinase activity during development. In addition to kinase activity, MEKK4 protein expression is also important in development. MEKK4 is highly expressed in the developing neuroepithelium and MEKK4 knockout mice display neural tube defects resulting in exencephaly and spina bifida (Chi et al., 2005). MEKK4 knockout mice also display a congenital malformation of the cerebral cortex and MEKK4 RNA interference impairs neuronal cell migration (Sarkisian et al., 2006).

Human MAP3K4 catalytic activity is activated by binding of GADD45 to the amino-terminal domain of MTK1 (Takekawa and Saito, 1998). In contrast, when the amino- and carboxyl-terminal domains of MTKs associate, this interaction is auto-inhibitory, blocking kinase activity. GADD45 association with MTK1 causes dissociation of the MTK1 amino-terminal and carboxyl-terminal domains leading to dimerization, auto-phosphorylation and activation of MTK1 (Miyake et al., 2007). Human MAP3K4 (MTK1) and the mouse homolog (MEKK4) regulate MKK6, which is upstream of stress activated p38 MAPK (Mita et al., 2002; Takekawa et al., 1997; Takekawa and Saito, 1998). In addition, stress induced activation of MEKK4 leads to activation of MEK4/7 and JNK (Abell et al., 2007).

Receptor tyrosine kinases (RTK's) and the growth factors that regulate them, such as heregulin (HRG) are often over-expressed in breast cancer cells (Kraus et al., 1989; Lemmon, 2003; Muller et al., 1988; Yarden and Sliwkowski, 2001), leading to activation of ERK1/2 activity, cell cycle progression (Fiddes et al., 1998) and cell migration (Krueger et al., 2001; Wu et al., 2008). The human epidermal growth factor receptors (HER) 1-4 are required for cell proliferation and differentiation during development (Darcy et al., 2000; Eccles, 2011). HER2 is an orphan receptor with no known ligand. HER2 can form a heterodimer with EGFR, HER3 or HER4 and is often over-expressed in breast cancer (Menard et al., 2000). HER4 expression correlates with favorable prognosis, while EGFR, HER2 and HER3 correlate with poor prognosis in breast cancer patients (Fuchs et al., 2006). The growth factor, heregulin, is a ligand for HER3 and HER4, however HER3 is not kinase active and requires hetero-dimerization with either EGFR, HER2 or HER4 for activity (Carraway et al., 1994; Lyne et al., 1997; Stern, 2008). Furthermore, HER2/HER3 is the preferred heterodimer for heregulin and produces strong mitogenic signaling that is linked to cancer (Alroy and Yarden, 1997; Amin et al., 2012; Stern, 2008; Way and Lin, 2005).

HER2 over expression in estrogen positive cells is associated with tamoxifin drug resistance in breast cancer (Kurokawa and Arteaga, 2003; Kurokawa et al., 2000; Shou et al., 2004). The drugs trastuzumab and lapatinib show high efficacy with HER2 positive patients, however drug resistance still persists (Bedard et al., 2009; Engel and Kaklamani, 2007; Esteva and Pusztai, 2005; Scaltriti et al., 2009; Stern, 2012). HER3 protein expression was shown to be up-regulated with lapatinib treatment, compensating for HER2 inhibition, and HER3 phosphorylation occurred by residual HER2 expression limiting the efficacy of lapatinib treatment (Garrett et al., 2011). Therefore, HER3 over-expression and recovery of phosphorylation appears to be a compensatory mechanism in response to drug targeting of HER2. HER3 requires the catalytic activity of other members of the HER family for phosphorylation. A unique feature of HER3 is the six YXXM binding motifs that when phosphorylated function as recruitment sites for the SH2 domain of p85 of phosphoinositide kinase 3 (PI3K) leading to increased cell motility, invasion and metastasis (Hellyer et al., 1998).

Cell migration requires actin polymerization and intracellular coordination of actin binding proteins, which are regulated by HER2 and downstream signaling proteins (Feldner and Brandt, 2002). For example, heregulin stimulation of breast cancer cells enhances the conversion of globular actin (g-actin) to filamentous actin (f-actin) increasing cell migration (Adam et al., 1998; Hijazi et al., 2000). Additionally, HER3 is regulated by HRG stimulation through HER2 kinase activity, which links HER3 to actin cytoskeletal reorganization and cell migration. Ssk2p is an example of an actin binding protein and is a homolog of MTK1. Ssk2p has an actin interacting region (AIR) that is required for actin cytoskeleton recovery after osmotic stress (Yuzyuk et al., 2002). Despite the evidence for Ssk2p involvement in actin cytoskeletal reorganization, a link between MTK1 and actin has not been established in mammalian cells. Furthermore, even though HER2 and HER3 are involved in actin reorganization and cell migration, MTK1 has not been identified in this signaling process.

Cancer cells have increased glycolytic metabolism leading to acid loading and excess protons are excreted by up-regulating proton transporters (Stock et al., 2005). Heregulin stimulation of breast cancer cells leads to extracellular acidification of media that is dependent on HER2/HER3 activity (Chan et al., 1995). Additionally, extracellular acidification affects cell migration and invasion (Kim et al., 2007; Sennoune et al., 2004). For instance, human melanoma cells treated with acidic media excrete proteases required for migration and are more invasive (Rofstad et al., 2006). With regard to HER2/HER3 signaling, although many signaling proteins have been linked to these receptors it is not clear how HRG regulates proton transporters.

Proteins that function in the MTK1 pathway have not been fully characterized nor has the regulation of MTK1 kinase activity. Previously, the present inventors have shown regulation of mouse MAP3K4 (MEKK4) to be through activation of the IFNγ cytokine receptor (Halfter et al., 2005) and the GPCR for angiotensin II (Derbyshire et al., 2005). Some aspects of the invention are based on the investigation by the present inventor on whether MTK1 is also regulated by the activation of RTK's in MCF-7 and T-47D epithelial breast cancer cells. The present inventors have discovered that the recruitment of MTK1 occurs with only activated HER3 in response to HRG in both MCF-7 and T-47D cells. It was also shown that MTK1 is also required for HRG induced cell migration in MCF-7 breast cancer cells through the HER2/HER3 heterodimer. Additionally, HRG induced association of MTK1 with p85 of PI3K, likely via phosphoHER3. It has been reported that HRG stimulation leads to extracellular acidification (Chan et al., 1995) an event that is linked to cancer cell migration (Stock et al., 2005; Stock and Schwab, 2009). The present inventors have shown that knockdown of MTK1 inhibited HRG-induced extracellular acidification and cell migration. Furthermore, pre-treatment of MCF-7 cells with the HER2 kinase inhibitor lapatinib inhibited association of MTK1 and HER3. MTK1 also associated with actin through the actin interacting region (AIR) and disruption of the actin cytoskeleton using cytochalasin D inhibited MTK1 and HER3 association. Thus, it is believed that MTK1 is an integral signaling protein downstream of activated HER2 and HER3 and is required for acidification of the extracellular environment and cell migration.

Other aspects of the invention describe the identification of the actin-interacting region of MTK1 (MAP Three Kinase 1) as a region consisting of 40 amino acids (amino acids 264-303) that is required for the activation of HER3/ErbB3 by the hormone, heregulin. As described above, HER3 is a receptor tyrosine kinase that lacks a catalytically active kinase domain but is active within the cell because HER3/ErbB3 forms a heterodimer with HER2/ErbB2. A subset of breast tumors as well as ovarian, gastric, and salivary cancers, owe their etiology to over-expression of HER2/ErbB2. In addition, mutations in the catalytic domain of HER2/ErbB2 have been identified in patients with non-small lung carcinoma. Although HER3/ErbB3 lacks a functional catalytic domain, HER3/ErbB3 is phosphorylated on a number of tyrosine residues with HER2/ErbB2 responsible for many of those phosphorylation events. Importantly once phosphorylated, these phosphotyrosines function as recruitment sites for other intracellular signaling proteins such as phosphatidylinositol 3-kinase (PI3-K), which is recognized in providing cancer with a survival advantage when compared to their normal cellular counterparts. The HER3/ErbB3 receptor is reported to have four canonical PI3-K recruitment sites, thus activation of HER3/ErbB3 in unique cancer cells is likely to confer those cells with a selective a survival advantage.

The HER2/ErbB2 receptor has been identified as a molecular target in cancer chemotherapy. A small molecule tyrosine kinase inhibitor (i.e., Lapatinib) and monoclonal antibodies (i.e., Trastuzumab and Pertuzumab) have been used in patients to treat various cancers. However, not all patients respond to these drugs or those patients that do respond eventually develop resistance to the drugs. Since the HER2/ErbB2 receptor functions as a partner with HER3/ErbB3 receptor, using the HER3/ErbB3 receptor as a molecular target is a viable alternative, but HER3/ErbB3 has a non-functional catalytic domain so using small molecule tyrosine kinase inhibitors as an approach to target specifically HER3/ErbB3 is believed to be not suitable in treating cancer.

Some aspects of the invention are based on targeting the HER3/ErbB3 receptor, but indirectly because MTK1 associates with only the activated form of HER3/ErbB3. In particular, the present inventors have identified the actin interacting region of MTK1 as mediating the interaction between MTK1 and the HER3/ErbB3 receptor. In some embodiments of the invention, the 40 amino acids within MTK1 (264-303) that constitute the actin-interacting region is used as a means to disrupt the interaction between MTK1 and the HER3/ErbB3 receptor. Thus, some aspects of the invention involve targeted disruption of the interaction between MTK1 and actin as an indirect approach to prevent the activation of ERK1/2 by HER3/ErbB3.

Activation of MTK1 in response to heregulin is required for cellular proliferation. Thus, inhibition of the interaction between MTK1 and HER3/ErbB3 is a novel approach to inhibit cellular proliferation in cells expressing the HER2/ErbB2-HER3/ErbB3 receptor heterodimer. The present inventors have found that a physical interaction occurs between MTK1 and HER3/ErbB3 but MTK1 does not interact with the HER2/ErbB2 tyrosine kinase receptor. In addition, MTK1 does not interact with the insulin receptor when cells are stimulated with insulin, the PDGF receptor when cells are stimulated with PDGF, or the EGF receptor (HER1/ErbB1) when cells are stimulated with EGF. Thus, the heregulin-dependent interaction between MTK1 and the HER3/ErbB3 receptor is unique among the receptor tyrosine kinase family.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Cell Culture and Treatments

HEK-293, T-47D and MDA-MB-231 cells were cultured in Dulbecco's modified Eagles medium with high glucose (DMEM) pH 7.4, supplemented with 10% fetal bovine serum (FBS) and 1% penicillin-streptomycin. MCF-7 cells were maintained in the same media as T-47D cells and supplemented additionally with 10 μg/ml insulin. Prior to experimental procedures, cells were cultured in DMEM supplemented only with 1% penicillin-streptomycin for 16 hrs. Cells were stimulated with 10 nM heregulin-β1 (HRG) EGF-Domain (Millipore Cat #01-201) for 12 minutes unless otherwise indicated, EGF 3.3 nM for 12 minutes, 0.3 M sorbitol for 30 minutes or vehicle (30% glycerol in 1× phosphate buffered saline pH 7.4) for 12 minutes. Pre-treatment with 250 nM lapatinib was performed during serum starvation for 16 hours unless otherwise indicated. Cells were treated with 1 μg/ml Cytochalasin D for 30 minutes prior to addition of HRG.

Western Blotting and Antibodies.

MCF-7 or T-47D cells were lysed in lysis buffer (70 mM β-glycerol phosphate, 1 mM EGTA, 1 mM dithiothreitol, 2 mM MgCl₂ and 0.5% Triton X-100) with protease inhibitors: 0.5 mM phenylmethylsulfonyl fluoride, 127.4 KIU/ml aprotinin (Calbiochem Cat #616399), 10 μM leupeptin and with 0.5 mM sodium orthovanadate. Proteins were resolved by 5%-12.5% gradient SDS-PAGE and transferred onto Protran 0.45 μm nitrocellulose blotting membrane (BioExpress Cat #F-3120-7). Membranes were blocked with 5% non-fat dry milk in 25 mM Tris-HCl, pH 7.4, 137 mM NaCl, 2.7 mM KCl and 0.15% Tween 20 (TBS-T). Immunostaining was performed in 5% non-fat dry milk in TBS-T and detected using chemiluminescence reagent (100 mM Tris pH 8.5, 250 mM luminol, 92 mM p-coumaric acid and 0.018% H₂O₂). Images were obtained using ChemiDoc™ XRS+ (BIO-RAD) and quantification was performed with Image Lab Software. After the initial immunoblots were performed, the nitrocellulose membranes were stripped at 56° C. for 1 to hour using membrane stripping buffer (12.5 mM Tris pH 6.8, 2% SDS, 0.7%(3-mercaptoethanol) to remove primary and secondary antibody. Membranes were re-imaged before additional immunoblots to ensure stripping was complete. Subsequent immunoblots were then performed the same way as described above. Antibodies were purchased from Cell Signaling (anti-mouse HRP-conjugated #7076S; anti-rabbit HRP-conjugated #7074S), Millipore (phosphotyrosine mouse monoclonal Clone 4G10 #05-321), Epitomics (EGFR #1902-1, HER2 #2064-1, HER3 #1186-1, HER4 #2218-1 and HER3 pY1289 #2526-1 rabbit monoclonal antibodies), Santa Cruz Biotechnology (PI3-Kinase p85α mouse monoclonal #sc-1637), Thermo Scientific (actin mouse monoclonal #MA1-744), Sigma (Anti-FLAG mouse monoclonal #F1804) and MTK1 antibodies used were developed as we previously described (Derbyshire et al., 2005). All commercial antibodies were used according to manufacturer recommendations.

Immunoprecipitation Experiments.

HEK-293, MCF-7, T-47D or MDA-MB-231 cells were stimulated with 10 nM HRG for 12 minutes, 0.3 M sorbitol for 30 minutes, 3.3 nM EGF for 12 minutes, 10 μg/ml insulin for 30 minutes or vehicle, unless otherwise indicated. The cells were then washed twice with ice-cold 1× phosphate buffered saline pH 7.4. The cells were then lysed in lysis buffer and 2 mg of cell extract was immunoprecipitated for 1 hour at 4° C. with 10 μg rabbit anti-MTK1 polyclonal antibody (Derbyshire et al., 2005); 4 μg mouse phosphotyrosine antibody (Millipore Clone 4G10 #05-321) or 20 μA anti-FLAG beads (Clontech #635686). Immune complexes recovered using protein A-Sepharose beads (Sigma Cat# P3391) were washed twice with ice-cold lysis buffer and denatured with Laemmli sample buffer. Proteins were separated by 5-12.5% gradient SDS-PAGE and transferred to nitrocellulose membranes that were then subjected to immunoblot analysis as indicated.

Cell Proliferation and Media pH Measurements.

MCF-7 and T-47D cells were seeded in complete growth media at a density of 0.4×10⁶ cells per well in 12-well plates (VWR #62406-165) for proliferation assays and 0.7×10⁶ cells per well for pH measurements. Cells were allowed to adhere for 16 hours, followed by 4 hour serum starvation in DMEM, 1% penicillin-streptomycin without fetal bovine serum. The cells were then stimulated with 10 nM HRG, 3.3 nM EGF, 250 nM lapatinib (lapatinib pre-treatment was started at time of serum starvation) or vehicle for 24 hours in DMEM with 0.5% fetal bovine serum, 1.0% penicillin/streptomycin and 10 μg/ml human recombinant insulin in MCF-7 cells. Each condition was performed in triplicate and repeated a minimum of 3 times. After 24 hours, the cells were counted using a TC-10 Bio-Rad automated cell counter and the media pH was recorded using a pH meter probe. Data shown for each condition represents n=3. A two sided student t-test with standard deviation were used to perform statistical analyses on proliferation rates and media pH measurements. Statistical significance was determined by a p-value ≦0.05.

siRNA Knockdown Experiments.

MCF-7 cells were seeded (onto 10 cm cell culture plates, VWR #353003) at a density of 5.0×10⁶ cells and allowed to adhere for 16 hours. The cells were then transfected with 434 pmol of siRNA specific for MTK1 (Thermo Scientific ON-TARGETplus SMARTpool, Human MAP3K4 (4216) Cat #L-003789-00-0005) or non-targeting (NS) (ON-TARGETplus Non-targeting Pool Cat #D-001810-10-05) as control using lipofectamine 2000 according to manufacture recommendations. The cells were transfected with siRNA a second time 24 hours later using lipofectamine 2000 to enhance knockdown efficiency. Twenty-four hours after the second transfection the cells were detached using 0.25% trypsin (Invitrogen #15050-065). The cells were counted and seeded onto 12 well plates (VWR #62406-165) at a density of 0.7×10⁶ cells per well for pH measurements and scratch assays or at density of 0.4×10⁶ cells per well for proliferation assays. MTK1 knockdown efficiency was determined by immunoblot analysis using 150 μg whole cell lysate at 120 hours post-secondary transfection. Each condition was performed in triplicate and repeated a minimum of 3 times. Data shown for each condition represents n=3. Statistical analysis was performed as described above.

Scratch Assay Experiments.

Scratch assays were performed in 12-well plates using MCF-7 cells at a density of 0.7×10⁶ cells per well. The cells were allowed to adhere for 16 hours followed by 4 hour serum starvation in DMEM. The surface area of the cells was scratched using a 10 μl pipette tip (VWR #89140-164) and the cells were then washed twice with 1× phosphate buffered saline pH 7.4. The cells were then stimulated with 10 nM HRG or vehicle, pretreated with or without 250 nM lapatinib (lapatinib pre-treatment was started at the time of serum starvation). Once scratches were performed and HRG stimulation was started, this was considered time zero and images were acquired using a 12.2 megapixel digital camera (GE model # W1200) visualized through a Leica Microsystems inverted microscope (model # DM IL) at 10× magnification. Additional images were taken at 24 and 48 hours and measurements were taken from 24 hours and 48 hours and compared to time zero for each condition. Each condition was performed in triplicate and repeated a minimum of 3 times. Data shown for each condition represents n=3. Statistical analysis was performed as described above.

Results: Association of a 180 kDa Tyrosine Phosphorylated Protein with MTK1.

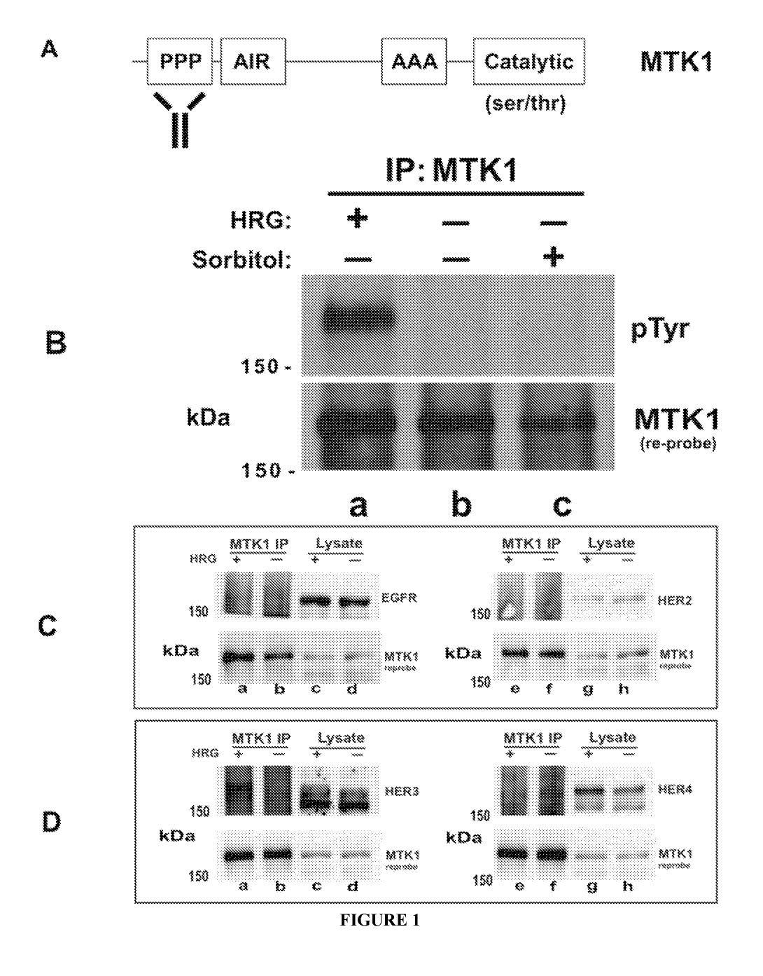

To investigate RTK regulation of MTK1, MCF-7 and T-47D breast cancer epithelial cells, which express moderate levels of the ErbB RTK receptors 1-4 (Beerli and Hynes, 1996; Carraway et al., 1994), were selected. Heregulin β1 (HRG) was used to stimulate these cells because HER3 and HER4 bind HRG allowing homo and hetero dimerization between HER1-4 (Aguilar et al., 1999; Carraway and Cantley, 1994; Neve et al., 2002). MCF-7 or T-47D cells were stimulated with HRG or sorbitol. Sorbitol was used to induce osmotic stress and activate MTK1, since MTK1 is known to function in the p38 MAP kinase pathway (Abell et al., 2007; Aissouni et al., 2005; Takekawa and Saito, 1998). After stimulation, MTK1 was immunoprecipitated with MTK1 antibody that recognizes the amino-terminal proline-rich region of MTK1 (FIG. 1A). Immunoprecipitated MTK1 and associated proteins were resolved by SDS-PAGE, followed by transfer to nitrocellulose membrane and immunoblotting for tyrosine phosphorylation (pTyr). A tyrosine phosphorylated protein co-immunoprecipitated with MTK1 in response to HRG stimulation (FIG. 1B, top panel, lane a), which was not present in cells stimulated with vehicle or sorbitol (FIG. 1B, top panel, lanes b and c). The tyrosine phosphorylated protein had a molecular weight of 180 kDa, which is approximately the same molecular weight as MTK1. This result suggested that MTK1 is phosphorylated on tyrosine in response to HRG stimulation, or that a HER family member co-precipitated with MTK1.

HER3 Associates with MTK1 in Response to HRG Stimulation.

The identity of the 180 kDa protein was investigated. HER1-4 were candidate proteins for tyrosine phosphorylation and association with MTK1, since HER3 and HER4 bind HRG and can trigger homo and hetero-dimerization of these four receptors (Britsch, 2007; Yarden and Sliwkowski, 2001). T-47D cells were stimulated with HRG and cell extracts were prepared in which MTK1 was immunoprecipitated in four different samples. The precipitated proteins were resolved by SDS-PAGE and immunoblotted for HER1-4 to identify which of the HER proteins associates with MTK1. HER3 was identified as associating with MTK1 in response to HRG stimulation (FIG. 1D, top panel, lane a). The fact that immunoblotted HER3 from the immunoprecipitation migrated slower than the proteins from the cell lysates suggested that phosphorylated HER3 preferentially interacted with MTK1. EGFR and HER2 did not associate with MTK1 in response to HRG or vehicle stimulation (FIG. 1C, top panel, lanes a & b, and lanes e & f). Moreover, HER4 did not associated with MTK1 in response to HRG (FIG. 1D, top panel, lanes e & f), even though HER4 binds HRG (Plowman et al., 1993). Immunoprecipitation of MTK1 was validated for each experimental condition by immunoblotting for MTK1 (FIG. 1C and FIG. 1D, bottom panels).

MTK1 is not Tyrosine Phosphorylated in Response to HRG.

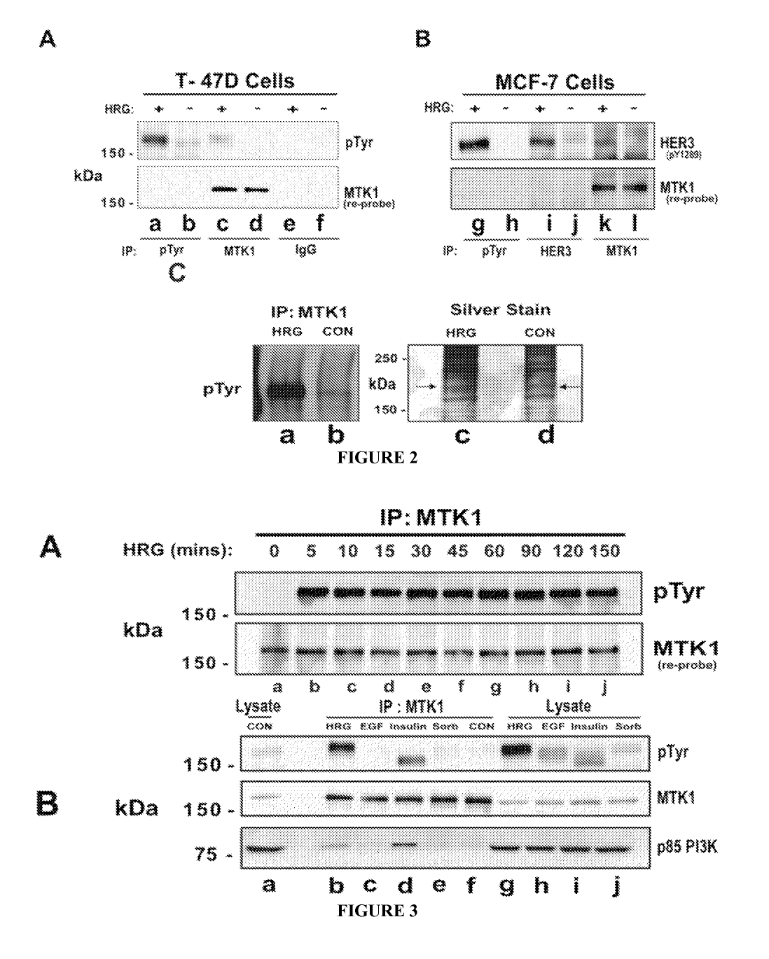

Experimental results suggested that HER3 was the tyrosine phosphorylated protein that interacts with MTK1. However, the results did not exclude the possibility that MTK1 was phosphorylated on tyrosine especially since MTK1 was immunoprecipitated, and it has a similar molecular mass as the tyrosine phosphorylated protein. If MTK1 was tyrosine phosphorylated, it should be possible to immunoprecipitate MTK1 with an antibody specific for phospho-tyrosine. T-47D cells were stimulated with HRG and an immunoprecipitation was performed with monoclonal antibody 4G10 that recognizes tyrosine phosphorylation. A protein of 180 kDa was immunoprecipitated with the 4G10 monoclonal antibody and the protein was phosphorylated on tyrosine (FIG. 2A, top panel, lane a). Similarly, a 180 kDa tyrosine phosphorylated protein was present in the MTK1 immunoprecipitation (FIG. 2A, top panel, lane c). When normal rabbit IgG was used as a control during the immunoprecipitation, no tyrosine phosphorylated proteins were detected in response to HRG (FIG. 2A. top panel, lane e). The membrane was stripped and immunoblotted for MTK1. The results revealed that MTK1 was not present in the phosphotyrosine immunoprecipitation (FIG. 2A, bottom panel, lanes a & b), which indicated that MTK1 is not tyrosine phosphorylated in response to HRG. As expected, MTK1 was present in the MTK1 immunoprecipitation (FIG. 2A, bottom panel, lanes c & d). Additionally, normal rabbit IgG did not immunoprecipitate MTK1 (FIG. 2A, bottom panel, lanes e & f).

PhosphoHER3 Associates with MTK1 in Response to HRG.

HER3 has a molecular mass of 180 kDa and among many phosphotyrosines there are several YXXM motifs that are phosphorylated in response to HRG (Hellyer et al., 1998). There are commercial phospho-antibodies that recognize several of these phosphorylation sites, one of which is pY1289. In order to determine if MCF-7 cells behave similarly to T-47D cells, MCF-7 cells were used in the next experiment. MCF-7 cells were stimulated with HRG and an immunoprecipitation was performed with monoclonal antibody 4G10. HER3 was immunoprecipitated with the 4G10 monoclonal antibody and was phosphorylated on Y1289 (FIG. 2B, top panel, lane g). Similarly when a HER3 antibody was used for immunoprecipitation, phosphoHER3 was detected with the pY1289 phospho antibody (FIG. 2B, top panel, lane i). Additionally, HER3 co-precipitated with MTK1 when the immunoprecipitation was performed with MTK1 antibody and HER3 was tyrosine phosphorylated on Y1289 (FIG. 2B, top panel, lane k). The membrane was stripped, immunoblotted for MTK1. No MTK1 was detected in the phosphotyrosine immunoprecipitation (FIG. 2B, bottom panel, lanes g & h). These results demonstrate that HRG regulates the association of MTK1 with activated HER3 and that MTK1 is not phosphorylated on tyrosine in both T-47D and MCF-7 breast cancer cells.

The association between MTK1 and HER3 in response to HRG stimulation was further characterized. A series of six MTK1 immunoprecipitations was performed in which 2 mg of cell extract was used for each immunoprecipitation. After washing the immunoprecipitations with lysis buffer, the samples were pooled and resolved in one lane by SDS-PAGE followed by silver staining Silver stained proteins were aligned with a replicate experiment that was immunoblotted with phosphotyrosine antibody (FIG. 2C, lane a). The area of the gel aligning with the 180 kDa tyrosine phosphorylated protein was excised (FIG. 2C, lanes c & d). The proteins were digested with trypsin, separated and analyzed via Orbitrap LC-MS. HER3 was identified as associating with MTK1 in response to HRG with 26% of the HER3 protein sequenced and several of the peptides identified with a 99.9% degree of confidence. Several of these peptides were sequenced multiple times increasing confidence for positive identification of HER3. Proteomic analysis did not identify HER3 as associating with MTK1 in response to vehicle stimulation. This result is consistent with immunoblot analysis which suggests that the association between HER3 and MTK1 is not constitutive, but requires HRG stimulation.

Although HER3 peptides were not identified from the MTK1 immunoprecipitation of vehicle stimulated MCF-7 cells, MTK1 peptides were identified because MTK1 and HER3 migrate at similar positions by SDS-PAGE (Supplemental FIG. 1). MTK1 peptides were identified from both the HRG and vehicle stimulated conditions. A total of 56% of the MTK1 protein was identified as tryptic peptides with a 99.9% degree of confidence for positive identification of MTK1. The identification of MTK1 peptides and not HER3 peptides from the vehicle stimulated sample indicates that the lack of HER3 peptides in this sample was not a technical artifact. In addition, there were no tyrosine phosphorylated peptides identified for MTK1 in either HRG or vehicle stimulated conditions which is consistent with the immunoblotting data.

A time course experiment was performed in which MCF-7 cells were stimulated with HRG for as long as 150 minutes. In other experiments, the tyrosine phosphorylation of the 180 kDa protein associating with MTK1 occurred within 30 seconds (data not shown). In longer time course experiments, the tyrosine phosphorylated protein that co-immunoprecipitated with MTK1 was observed from 5 for up to 150 minutes after HRG stimulation (FIG. 3A, upper panel, lanes b-j). In HRG washout experiments where HRG was washed out after each time point, the tyrosine phosphorylation was more transient (data not shown). These experiments demonstrate that constant HRG exposure results in continued HER3 activation and association with MTK1. This result may be significant in breast cancer cases in which HRG is over-expressed.

HER3 and MTK1 Association is Specific to HRG Stimulation.

In order to determine whether other receptor tyrosine kinases interacted with MTK1, T-47D cells were stimulated with EGF or insulin and then MTK1 was immunoprecipitated as described above. As a positive control, cells were stimulated with HRG. Immunoblot analysis using 4G10 mouse monoclonal antibody revealed a protein of approximately 150 kDa that was tyrosine phosphorylated in response to insulin (FIG. 3B, top panel, lane d). No tyrosine phosphorylated proteins immunoprecipitated with MTK1 after stimulation with EGF, sorbitol or vehicle (FIG. 3B, top panel, lanes c, e & f). Since EGF can activate the EGFR and HER2, the lack of tyrosine phosphorylated proteins indicates that neither EGFR nor HER2 interact with MTK1, while HRG selectively activates HER3 to interact with MTK1 (lane b). The ˜150 kDa tyrosine phosphorylated protein that immunoprecipitated with MTK1 in response to insulin was later identified as insulin receptor substrate-1 (IRS-1; data not shown). The membrane was stripped and immunoblotted for MTK1 (FIG. 3B, middle panel). To establish stimulation efficacy, lysates from vehicle, HRG, EGF, insulin and sorbitol were immunoblotted with G410 mouse monoclonal antibody (FIG. 3B, top panel lanes a & g-j).

p85 of PI3K Associates with MTK1.

HER3 has several YXXM motifs which, when phosphorylated, function as recruitment sites for the SH2 domain of the p85 regulatory subunit of PI3K (Hellyer et al., 1998). Therefore, it was possible that p85 would co-precipitate with MTK1 in response to HRG. p85 of PI3K was identified as immunoprecipitating with MTK1 in response to HRG stimulation (FIG. 3B, bottom panel, lane b). Additionally, p85 was also shown to immunoprecipitate with MTK1 in response to insulin stimulation, likely through an interaction with IRS (FIG. 3B, bottom panel, lane d). These results show that the HER3/MTK1 complex also includes PI3K suggesting that the HER3/MTK1 complex utilizes 3′ phosphorylated inositol phosphates as part of the signaling mechanism.

HER2 is Required for Co-Immunoprecipitation of Phosphorylated HER3 with MTK1.

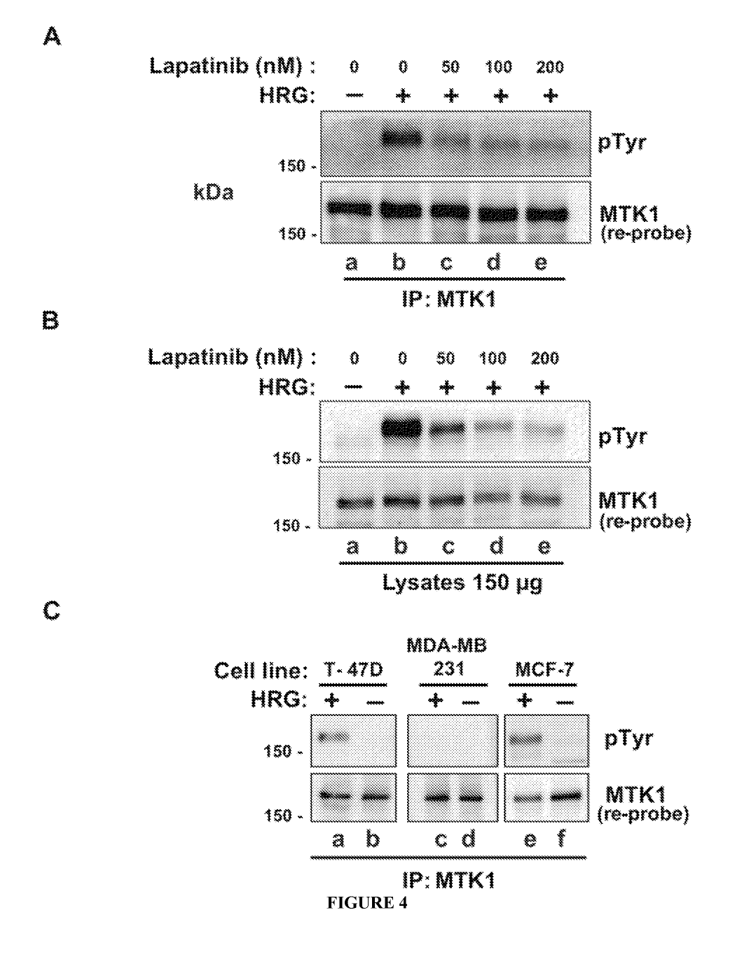

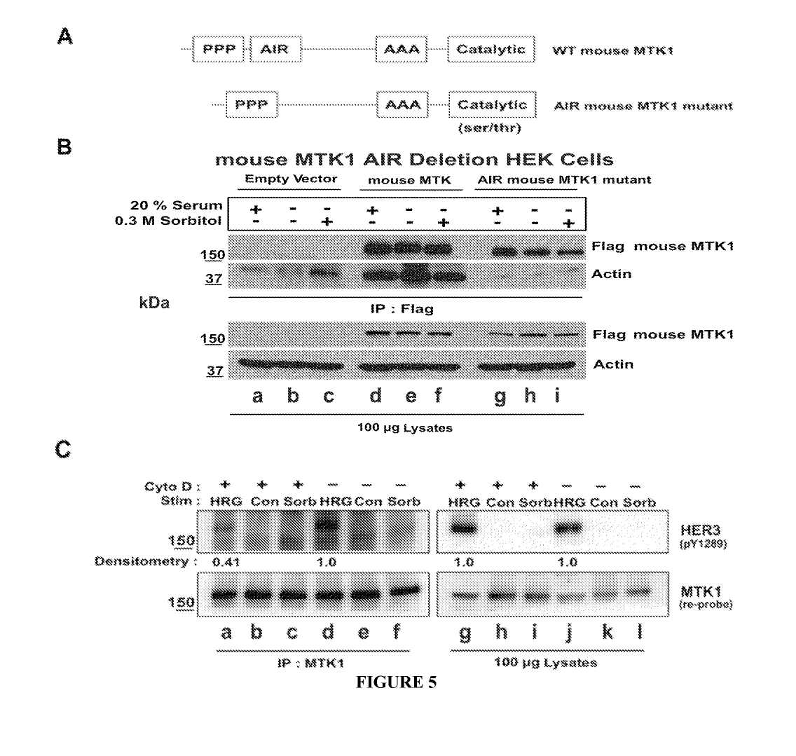

HER3 does not have intrinsic kinase activity and is phosphorylated by hetero-dimerization with either HER1, 2 or 4 (Stern, 2008). HER2, HER3 and HER4 are tyrosine phosphorylated in MCF-7 cells stimulated with HRG. Thus, HER2 and HER4 are possible candidates for phosphorylating HER3, since HER2/HER3 and HER3/HER4 heterodimers are formed in response to HRG. Since HER2 and HER3 are preferred heterodimers (Way and Lin, 2005), HER2 was hypothesized as the most likely candidate for phosphorylation of HER3. MCF-7 cells were pretreated with lapatinib to inhibit the kinase activity of HER2 and then stimulated with HRG followed by immunoprecipitation of MTK1. Lapatinib has a Kᵢ of 13 nM for inhibition of HER2 (Wood et al., 2004). Lapatinib decreased the interaction between HER3 and MTK1 in a dose dependent manner as evidenced by decreased tyrosine phosphorylation of HER3 (FIG. 4A). These results indicate that HER2 catalytic activity is required for HER3 phosphorylation and subsequent interaction with MTK1 in response to HRG. The membrane was stripped and immunoblotted for MTK1 (FIG. 4A, bottom panel). Lysates from the same experiment show tyrosine phosphorylation of HER3 is decreased in response to lapatinib (FIG. 4B, top panel, lanes c-e).

MDA-MB-231 breast cancer cells were used to further characterize HER2 involvement in the formation of the HER3-MTK1 protein complex in response to HRG. MDA-MB-231 cells are a triple negative cell line and do not express estrogen receptor, progesterone receptor or HER2, but do express HER3 (Teixido et al., 2012). If HER2 is required for HER3 phosphorylation and association with MTK1, then the association of HER3 with MTK1 in MDA-MB-231 would not be observed. T-47D, MDA-MB-231 and MCF-7 cells were stimulated with HRG followed by immunoprecipitation of MTK1. Immunoblot analysis using 4G10 mouse monoclonal antibody revealed that a 180 kDa tyrosine phosphorylated protein immunoprecipitated with MTK1 in T-47D and MCF-7 cells dependent on HRG (FIG. 4C, top panel, lanes a & e). No tyrosine phosphorylated proteins immunoprecipitated with MTK1 from the MDA-MB-231 cells (FIG. 4C, top panel, lanes c & d), which strongly suggests that HER2 is required for formation of the HER3 MTK1 complex. The membranes were stripped and immunoblotted for MTK1 to demonstrate that MTK1 is expressed in MDA-MB-231 cells (FIG. 4C, bottom panel).

Actin Interacting Region (AIR) of MEKK4 is Required for Actin Association with MEKK4.

Human MTK1 and the mouse homolog, MEKK4, have similar homology to the yeast protein, Ssk2p (Bettinger and Amberg, 2007). Ssk2p has an actin interacting region (AIR) that is required for actin cytoskeleton recovery after osmotic stress (Yuzyuk et al., 2002). Alignment of the AIR of Ssk2p with MEKK4 showed 17/40 amino acids were identical between these proteins and 25/40 were highly conserved, suggesting that MEKK4/MTK1 might interact with actin (FIG. 5A). The AIR of MEKK4 consisting of amino acids 256-295 was deleted from the MEKK4 cDNA. Flag-tagged wild-type MEKK4 and flag-tagged AIR MEKK4 were transfected into HEK 293 cells. After 48 hours, the HEK cells were serum starved for 4 hours followed by stimulation with 20% fetal bovine serum or sorbitol as possible inducers of the MTK1-actin interaction. MEKK4 was immunoprecipitated with a flag antibody-coupled to beads that specifically recognizes the flag epitope. Actin did not associate with MEKK4 when the AIR was deleted (FIG. 5B, second panel, lanes g-i), while actin constitutively associated with wild-type MEKK4 independent of stimulation (FIG. 5B, second panel, lanes d-f). These results demonstrate that MEKK4/MTK1 behave like Ssk2p and interact with actin within a unique 40 amino acid region.

Disruption of the Actin Cytoskeleton Significantly Decreases the Association Between HER3 and MTK1.

HRG enhances the conversion of g-actin to f-actin leading to increased cell migration (Adam et al., 1998). Since HER3 is regulated by HRG, the association between MTK1 and HER3 that we observed may be facilitated by actin in response to HRG. To test this hypothesis, MCF-7 cells were pre-treated with cytochalasin D to disrupt actin polymerization and the formation of f-actin. The cells were then stimulated with HRG or sorbitol followed by immunoprecipitation of MTK1 and pY1289 HER3 immunoblots. In the presence of cytochalasin D, the interaction between HER3 and MTK1 was diminished by 60% (FIG. 5C, top panel, compare lanes a & d). Interestingly, phosphorylation of HER3 remained the same in the HRG stimulated lysates (FIG. 5C, top panel, lanes g & j). These data suggest that f-actin is required for the association between activated HER3 and MTK1 in response to HRG, but f-actin is not required for HRG-dependent activation of HER3.

MTK1 is Required for HRG-Induced Extracellular Acidification.

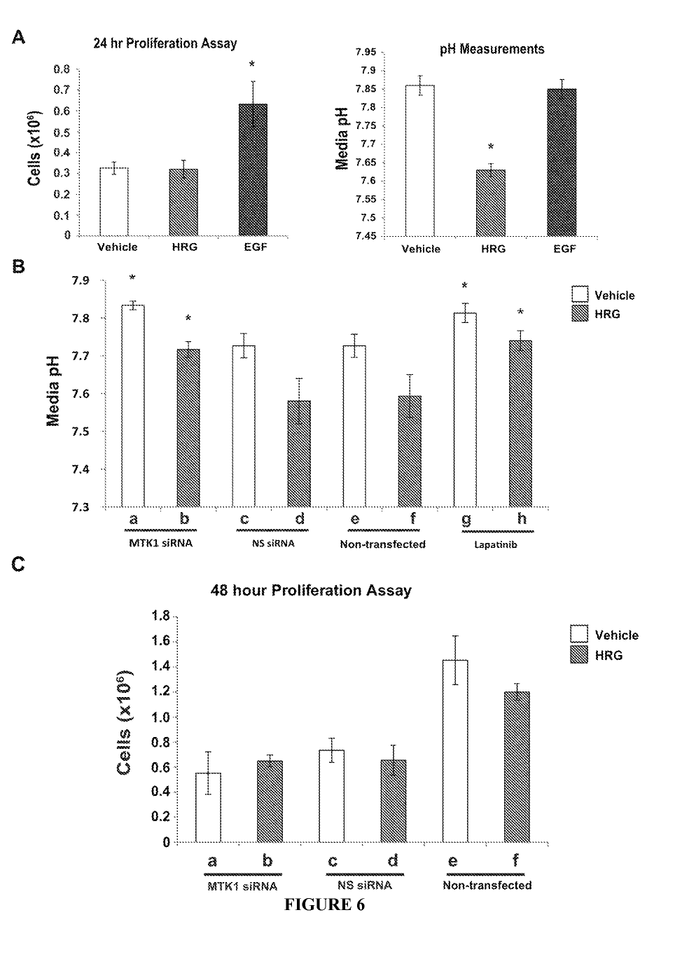

Since HRG is known to induce proliferation, invasion, migration and metastasis (Asrani et al., 2013; Atlas et al., 2003; Fiddes et al., 1995; Hernandez et al., 2009; Xue et al., 2006; Yang et al., 2008), it was necessary to identify the specific functional role of HRG in MCF-7 breast cancer cells. Assays were performed to determine if HRG stimulation leads to proliferation of MCF-7 cells. MCF-7 cells were stimulated with HRG or EGF and after 24 hours the cells were counted using a Bio-Rad TC-10 automated cell counter in the presence of trypan blue. EGF, unlike HRG, induced cell proliferation (FIG. 6A, left panel). There was no difference in cell viability between each condition (data not shown). However, there was a noticeable difference in the color of the DMEM media after 24 hours of HRG stimulation, which was not observed with EGF stimulation. Media from the HRG stimulated cells appeared orange, which is an indicator of increased acidity or decreased pH. Measurements of the media pH revealed that HRG stimulation led to extracellular acidification of the cell culture media when compared to vehicle (FIG. 6A, right panel). EGF stimulation did not induce extracellular acidification (FIG. 6A, right panel). Similarly, T-47D cells stimulated with HRG also had extracellular acidification independent of cell proliferation (data not shown).

MTK1 Knockdown Inhibits HRG Induced Extracellular Acidification.

HRG induced extracellular acidification and association of tyrosine phosphorylated HER3 with MTK1. These results suggest that MTK1 is required for HRG-induced proton excretion from the cell. To test this hypothesis MCF-7 cells were double transfected with siRNA that specifically targets MTK1 and non-specific (NS) siRNA as a control (double transfections were performed to increase transfection efficiency). The cells were stimulated with HRG and after 24 hours, media was collected and pH measurements were recorded with a pH meter probe. Knockdown of MTK1 inhibited HRG induced extracellular acidification when compared to NS siRNA and non-transfected cells (FIG. 6B, lanes b, d & f). In addition, pre-treatment of MCF-7 cells with lapatinib for 30 minutes prior to stimulation with HRG resulted in inhibition of HRG-induced extracellular acidification (FIG. 6B, lanes g & h) indicating a requirement for HER2 in the acidification response. Together, these results show that HER2 and MTK1 are required for HRG-induced extracellular acidification.

Knockdown of MTK1 could affect the proliferation rate resulting in fewer cells excreting protons. Therefore, proliferation assays were performed to rule out whether MTK1 knockdown had an effect on proliferation rates. MCF-7 cells were transfected with siRNA, as described previously and stimulated with HRG. After 48 hours the cells were counted using a Bio-Rad TC-10 automated cell counter. Knockdown of MTK1 did not have an effect on the proliferation rate when compared to NS siRNA knockdown (FIG. 6C, lanes a-d). Knockdown of MTK1 did not have an effect on viability either (data not shown). Since all of the experimental conditions shown in FIG. 6C started with the same amount of cells, it appears that transfection of MCF-7 cells did have an effect on proliferation rate when compared to non-transfected cells (FIG. 6C, lanes e & f). However, the decreased proliferation rate was independent of MTK1 siRNA but dependent on transfection, likely reflecting the difficulty associated with transfecting MCF-7 cells.

MTK1 is Required or HRG Induced Cell Migration.

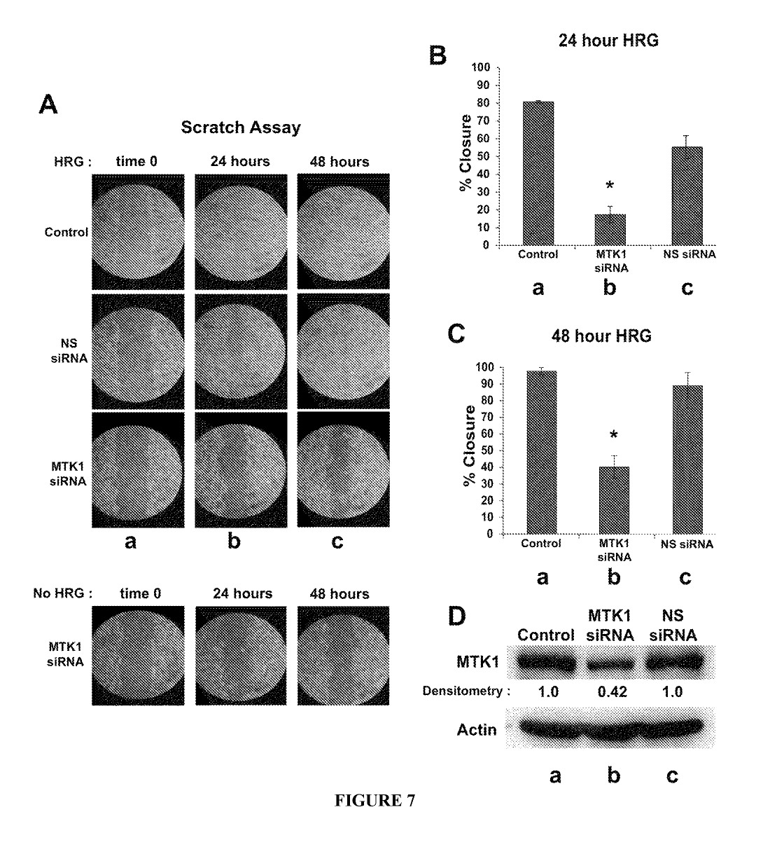

Cancer cells have increased glycolytic metabolism leading to acid loading and excess protons are excreted by up-regulating proton transporters (Stock et al., 2005). Migration is a possible functional response to HRG stimulation because acid excretion creates micro extracellular environments that are favorable for cell migration (Stock and Schwab, 2009). HRG is also linked to intracellular actin cytoskeletal reorganization that leads to increased migration (Adam et al., 1998). Furthermore, MTK1 may have a role in HRG induced cell migration since MTK1 knockdown blocks extracellular acidification. To determine whether MTK1 is required for cell migration, HRG-induced scratch assays were performed with and without MTK1 knockdown. MCF-7 cells were transfected with MTK1 and NS siRNA as described above. MCF-7 cell transfections were performed using 10 cm culture dishes and then the cells were transferred to a 12-well plate after a 24 hour post-transfection recovery period. The surface of the well was scratched followed by HRG stimulation and digital image acquisition at 0, 24 and 48 hours post scratch. After 24 hours of HRG stimulation, cells transfected with MTK1 siRNA closed the scratched area by approximately 20% while the cells transfected with NS siRNA had closed the scratched area by approximately 60% (FIG. 7A, column b). Additionally, MTK1 knockdown continued to severely impair cell migration into the scratched area after 48 hours (FIG. 7A, column c), while the cells transfected with NS siRNA had almost completely migrated into the scratched area, much like the non-transfected cells (FIG. 7A, top row). Statistical analysis revealed that MTK1 knockdown had a mean of 17.3%±4.6 cell migration (FIG. 7B, lane b) compared NS knockdown mean of 55.3%±6.4 (FIG. 7B, lane c). Similar results were observed at 48 hours with MTK1 knockdown cells migrating by a mean of 40.1%±6.8 (FIG. 7C, lane b) compared to NS knockdown migrating by a mean of 89%±7.8 (FIG. 7C, lane c). Very little cell migration was observed in cells not stimulated with HRG. Therefore, scratch assays of cells transfected with MTK1 siRNA or NS siRNA, and non-transfected cells all looked similar (only MTK1 siRNA is shown, FIG. 7A bottom panel). MTK1 protein expression levels were measured 48 hours after scratch by immunoblot analysis. MTK1 protein expression was knocked down by 58% compared to NS siRNA (FIG. 7D, top panel, lanes b & c). Actin was used as a loading control (FIG. 7D, bottom panel). Together these results demonstrate that HRG-induced cell migration requires MTK1.

Discussion:

Prior studies have used ectopic expression of MEKK4/MTK1 in yeast (Takekawa et al., 1997), over-expression of wild-type or dominant-negative mutants in mammalian cells (Gerwins et al., 1997; Kanungo et al., 2000; Mita et al., 2002; Stevens et al., 2006; Takekawa and Saito, 1998), or manipulation of MEKK4 genomic DNA (Abell et al., 2005; Sarkisian et al., 2006; Sun et al., 2011) as a means to study MEKK4/MTK1 function. In this study the present inventors have focused on the scaffolding properties of endogenous MTK1 and have identified a heregulin-dependent recruitment of tyrosine phosphorylated HER3 to MTK1. HER2 kinase activity is required for the formation of the HER3/MTK1 heterodimer, which is stable and involves only tyrosine phosphorylated HER3. These results have clinical implications in patients prescribed drugs like trastuzumab and lapatinib to inhibit HER2 activity, since these drugs will more than likely indirectly inhibit the activity of the phosphoHER3/MTK1 heterodimer. Studies by the present inventors have demonstrated that extracellular acidification and cell migration require MTK1. Thus, both normal and malignant biological processes dependent on extracellular acidification and cell migration would be inhibited in patients treated with trastuzumab or lapatinib.

It is clear that tyrosine phosphorylated HER3 preferentially associates with MTK1 as demonstrated when the hyper-phosphorylated, slower migrating form of HER3 co-immunoprecipitates with MTK1 (FIG. 1D, top panel, lane a). Consistent with the immunoblotting results, multiple peptides were identified by LC-MS/MS from heregulin-stimulated HER3, but no peptides were detected from the control sample, again demonstrating that only activated HER3 interacts with MTK1. These results show that a specific or multiple phosphotyrosines mediate the interaction between phosphoHER3 and MTK1. MTK1 has no known SH2 domains so a direct interaction between MTK1 and phosphoHER3 seems unlikely. However, Shc, PI3K, and Grb7 have SH2 domains and are examples of proteins that interact with specific phosphotyrosines on HER3 (Jones et al., 2006) and these proteins could mediate the interaction between MTK1 and phosphoHER3.

The HER2/HER3 heterodimer has been reported as an oncogenic unit that drives breast tumor cell proliferation. Although HER2 is the only catalytically active kinase within the heterodimer, HER3 is required for breast tumor formation (Garrett et al., 2011; Holbro et al., 2003; Vaught et al., 2012). Thus even though HER3 lacks tyrosine kinase activity and depends on HER2 for phosphorylation, the proteins that “decorate” phosphoHER3 are also mediators of HER2/HER3 tumorigenicity. The present inventors have discovered that HRG stimulation recruits MTK1 to HER3, which is sustained for up to 150 minutes indicating that sustained exposure to HRG results in continuous signaling through MTK1 and does not result in receptor down-regulation, consistent with previous findings demonstrating lack of receptor down-regulation with HER2 and HER3 (Baulida et al., 1996). The ability of HER2/HER3 to continue signaling through MTK1 in the presence of HRG have clinical implications for patients with HRG-driven tumors as opposed to tumors with HER2 gene amplification. In tumors with over-expression of HRG, an autocrine or paracrine loop may play a role in cellular transformation and tumor progression that does not require HER2 over-expression. Identification of the HER3 regulated MTK1 pathway provides a new molecular target for clinical intervention under conditions of normal levels of HER2/HER3 expression.

Whether MTK1 interacts with other RTK was also investigated. Although HRG also binds to HER4 (Hynes and Lane, 2005), phosphoHER4 does not appear to interact with MTK1 (FIG. 1D). EGF is also an activator of HER2 (Khazaie et al., 1993), but stimulation of MCF-7 cells did not result in the recruitment of EGFR or HER2, or any other tyrosine phosphorylated proteins that would migrate at the position of 150 kDa or greater (FIG. 3, lane c). Insulin stimulation of MCF-7 cells led to the recruitment of a 150 kDa tyrosine phosphorylated protein (FIG. 3B, lane d). This protein was identified as the insulin receptor substrate-1. When the insulin receptor is activated, IRS-1 associates with the insulin receptor and is tyrosine phosphorylated. IRS-1 helps attenuate insulin signaling in healthy individuals (Schmitz-Peiffer and Whitehead, 2003). However, IRS-1 is also associated with breast cancer and is characterized as a transforming oncogene (Chan and Lee, 2008). The interaction of MTK1 with HER3 and IRS-1 strongly links MTK1 to RTK signaling. Thus, the interaction between MTK1 and IRS-1 is also a new avenue in breast cancer therapy. Finally, stimulating MCF-7 cells with sorbitol did not result in the recruitment of phosphoHER3 with MTK1 indicating that the interaction between MTK1 and phosphoHER3 is not related to osmotic stress (FIG. 3B, lane e). In summary, the recruitment of MTK1 with phosphoHER3 appears to be unique to HRG binding to the HER2/HER3 heterodimer identifying an entirely new signaling pathway downstream of HER3.

The carboxyl-terminus of HER3 contains six YXXM motifs that when phosphorylated allow association of p85 of PI3K (Hellyer et al., 1998; Soltoff et al., 1994). Mutation of the YXXM motifs from tyrosine to phenylalanine inhibits HRG-induced migration suggesting that recruitment of PI3K with HER3 is important in mediating cell migration (Smirnova et al., 2012). Furthermore, inhibition of PI3K with PIK-75 inhibits cell motility and invasion (Smirnova et al., 2012). The present inventors have shown that knockdown of MTK1 also blocks HRG-induced migration (FIG. 7). It is tempting to speculate that a single mole of phosphoHER3 and MTK1 functions as a signaling complex to recruit as many as six moles of PI3K, leading to amplification of the cell migration signal. Thus even though MTK1 knockdown was not highly efficient, loss of 60% of MTK1 expression was sufficient to prevent HRG-dependent cell migration. Perhaps indicating that a threshold of PI3K activity cannot be mobilized to lamellipodia or other cellular regions actively involved in cell migration. Similarly in the presence of insulin, PI3K is recruited to MTK1 (FIG. 3B, lane d), likely due to the presence of IRS-1 which is known to associate with PI3K (Kooijman et al., 1995).

HER3/MTK1 association requires HER2 kinase activity, but HER2 does not remain in the complex. Lapatinib inhibits EGFR, HER2 and HER4 by binding to the ATP-binding pocket of these kinases, thereby inhibiting catalytic activity. Lapatinib shows a dose-dependent inhibition of EGFR with a Kᵢ=3 nM, HER2 with a Kᵢ=13 nM, and HER4 with a Kᵢ=347 nM (Wood et al., 2004). In a dose response experiment, pre-treatment of MCF-7 cells with 50 nM lapatinib effectively inhibited HRG-induced HER3 phosphorylation and association with MTK1 (FIG. 4). Since HER4 has a Kᵢ of 347 nM for lapatinib, EGFR and HER2 were the more likely candidates for lapatinib blockade of HER3 phosphorylation and subsequent MTK1/HER3 association. EGFR is expressed in MCF-7 and T-47D cells and can form heterodimers with HER3 (Eccles, 2011; Yarden et al., 1996). However, experiments comparing EGF and HRG showed that the EGFR is not activated in response to HRG (data not shown). Together, these results show that lapatinib inhibited HER2 and effectively inhibited HER3 phosphorylation and formation of the MTK1/HER3 heterodimer. Additionally in HER2 negative and HER3 positive MDA-MB-231 cells, HRG does not induce formation of the MTK1/HER3 dimer (FIG. 4C). Therefore, these data indicate that HER2 protein expression and kinase activity is required for HER3 phosphorylation and MTK1/HER3 association.

Cell migration requires actin polymerization and intracellular coordination of actin binding proteins, which are regulated by signal transduction and subsequent second messenger signaling (Feldner and Brandt, 2002). Migration is also an important part of cancer cell invasion and metastasis (Yamazaki et al., 2005). HER2 is linked to actin cytoskeletal reorganization in cancer (Feldner and Brandt, 2002) and heregulin stimulation of breast cancer cells enhances the conversion of g-actin to f-actin increasing cell migration (Adam et al., 1998; Hijazi et al., 2000), implicating HER3 with actin cytoskeletal reorganization. Additionally, MEKK4 has been shown by the present inventors to interact with actin through a 40 amino acid region referred to as the actin interacting region (AIR), which is also found in yeast Ssk2p (Yuzyuk et al., 2002). Deletion of the AIR within MEKK4 impairs the constitutive association between MEKK4 and actin (FIG. 5B). These results demonstrate a proximal localization of MEKK4 with the actin cytoskeleton. When MCF-7 cells were pre-treated with cytochalasin D to prevent actin polymerization, the interaction between MTK1 and HER3 was reduced by ˜60%, indicating a cytoskeletal connection involving actin linking MTK1 and HER3.

Although others have reported that HRG induces a proliferative response (Yang et al., 2008), the present inventors were unable to detect HRG-dependent proliferation in either T-47D (data not shown) or MCF-7 cells (FIG. 6A), although an EGF-dependent proliferative response was detected. As the cells were incubated with either HRG or EGF, it was noticed that the cell media developed a more orange color in the presence of HRG. When the pH was recorded, it was consistently observed that HRG caused an acidification of the extracellular medium while EGF had no effect. Given that HRG caused an association between MTK1 and HER3, siRNA was used to decrease the expression of MTK1 to determine whether MTK1 was involved in regulating extracellular pH. Although siRNA typically diminished MTK1 expression by only ˜60% in MCF-7 cells (see FIG. 7D for a representative immunoblot), the change in extracellular pH was always diminished in the absence of MTK1 (FIG. 6B, lanes a & c). These results demonstrate that EGF and HRG induce different responses in MCF-7 cells and that the EGF-dependent proliferative response does not correlate with acidification of the extracellular medium. Since HRG did not induce proliferation of MCF-7 cells, whether HRG induced cell migration was investigated using a scratch assay. After 24 hours of HRG exposure in the presence of NS siRNA, 60% of the cells migrated back into the scratched region (FIG. 7A). In contrast, only 20% of the cells migrated back into the scratch region after 24 hours in cells with MTK1 knockdown. These results demonstrate that HRG induces migration and not proliferation of MCF-7 cells, consistent with the results of Arsani et al. (Asrani et al., 2013). It was also shown that MTK1 is required to acidify the extracellular environment. The acidification of the extracellular environment likely helps optimize the enzymatic activity of matrix metalloproteinases or other enzymes needed for the migratory response.

MTK1 is not tyrosine phosphorylated in response to HRG stimulation of T-47D cells (FIG. 2A, lane a) or MCF-7 cells (FIG. 2A, lane g). However, the association between MTK1 and HER3 is dependent on HRG. Since MTK1 is not tyrosine phosphorylated, it is unlikely that MTK1 is a substrate for HER2. Experiments showed phosphorylation of serine 686 of HER3 (LERGEpSIEP) in response to HRG by sequencing using LC-MS/MS (FIG. 2B). Phosphorylation of HER3 at serine 686 has been previously reported, but the functional significance of this modification is not known (Daub et al., 2008). Earlier studies have shown MEKK4 functions as a serine/threonine kinase regulating MKK6, which is upstream of p38 (Gerwins et al., 1997; Mita et al., 2002; Takekawa et al., 1997; Takekawa and Saito, 1998). Experiments also showed that MTK1 functions as a scaffolding protein for phosphoHER3. However, since MTK1 is a serine/threonine kinase, it is possible that the catalytic activity of MTK1 is required to regulate changes in extracellular pH and cell migration.

HER2/HER3 heterodimerization has been well characterized, but the present disclosure shows for the first time that HRG stimulation results in the formation of a MTK1/HER3 heterodimer, which is sustained for up to 150 minutes in breast cancer cells. The present disclosure also demonstrates that HER2 kinase activity is needed for association of MTK1 with phosphoHER3. Furthermore, disruption of actin polymerization inhibits HRG induced MTK1/HER3 association, which suggests that f-actin is required to bring MTK1 and phosphoHER3 together linking MTK1 to the actin cytoskeleton. Additionally, MTK1 is required for HRG induced extracellular acidification and cell migration. In conclusion, over-expression of HER2 in breast cancer has been targeted with drugs like trastuzumab and lapatinib, however drug resistance or unresponsiveness persists among many patients. The link between HER3 up-regulation to HER2 drug resistance makes HER3 a desirable molecular target for anti-cancer drug development. However, the lack of HER3 catalytic activity has made HER3 somewhat less tractable as a drug target. By identifying the recruitment of MTK1 to phosphoHER3, the present disclosure shed new light onto the HER3 branch of the HER2/HER3 signaling tree.

### REFERENCES

Abell, A. N., D. A. Granger, and G. L. Johnson. 2007. MEKK4 stimulation of p38 and JNK activity is negatively regulated by GSK3beta. J Biol Chem. 282:30476-30484.
Abell, A. N., J. A. Rivera-Perez, B. D. Cuevas, M. T. Uhlik, S. Sather, N. L. Johnson, S. K. Minton, J. M. Lauder, A. M. Winter-Vann, K. Nakamura, T. Magnuson, R. R. Vaillancourt, L. E. Heasley, and G. L. Johnson. 2005. Ablation of MEKK4 kinase activity causes neurulation and skeletal patterning defects in the mouse embryo. Mol Cell Biol. 25:8948-8959.
Adam, L., R. Vadlamudi, S. B. Kondapaka, J. Chernoff, J. Mendelsohn, and R. Kumar. 1998. Heregulin regulates cytoskeletal reorganization and cell migration through the p21-activated kinase-1 via phosphatidylinositol-3 kinase. J Biol Chem. 273:28238-28246.
Aguilar, Z., R. W. Akita, R. S. Finn, B. L. Ramos, M. D. Pegram, F. F. Kabbinavar, R. J. Pietras, P. Pisacane, M. X. Sliwkowski, and D. J. Slamon. 1999. Biologic effects of heregulin/neu differentiation factor on normal and malignant human breast and ovarian epithelial cells. Oncogene. 18:6050-6062.
Aissouni, Y., G. Zapart, J. L. Iovanna, I. Dikic, and P. Soubeyran. 2005. CIN85 regulates the ability of MEKK4 to activate the p38 MAP kinase pathway. Biochem Biophys Res Commun. 338:808-814.
Alroy, I., and Y. Yarden. 1997. The ErbB signaling network in embryogenesis and oncogenesis: signal diversification through combinatorial ligand-receptor interactions. FEBS letters. 410:83-86.
Amin, D. N., N. Sergina, L. Lim, A. Goga, and M. M. Moasser. 2012. HER3 signalling is regulated through a multitude of redundant mechanisms in HER2-driven tumour cells. The Biochemical journal. 447:417-425.
Asrani, K., R. A. Keri, R. Galisteo, S. A. Brown, S. J. Morgan, A. Ghosh, N. L. Tran, and J. A. Winkles 2013. The HER2- and heregulin betal (HRG)-inducible TNFR superfamily member Fn14 promotes HRG-driven breast cancer cell migration, invasion, and MMP9 expression. Molecular cancer research: MCR. 11:393-404.
Atlas, E., M. Cardillo, I. Mehmi, H. Zahedkargaran, C. Tang, and R. Lupu. 2003. Heregulin is sufficient for the promotion of tumorigenicity and metastasis of breast cancer cells in vivo. Molecular cancer research: MCR. 1:165-175.
Baulida, J., M. H. Kraus, M. Alimandi, P. P. Di Fiore, and G. Carpenter. 1996. All ErbB receptors other than the epidermal growth factor receptor are endocytosis impaired. J Biol Chem. 271:5251-5257.
Bedard, P. L., E. de Azambuja, and F. Cardoso. 2009. Beyond trastuzumab: overcoming resistance to targeted HER-2 therapy in breast cancer. Current cancer drug targets. 9:148-162.
Beerli, R. R., and N. E. Hynes. 1996. Epidermal growth factor-related peptides activate distinct subsets of ErbB receptors and differ in their biological activities. J Biol Chem. 271:6071-6076.
Bettinger, B. T., and D. C. Amberg. 2007. The MEK kinases MEKK4/Ssk2p facilitate complexity in the stress signaling responses of diverse systems. J Cell Biochem. 101:34-43.
Britsch, S. 2007. The neuregulin-I/ErbB signaling system in development and disease. Advances in anatomy, embryology, and cell biology. 190:1-65.
Carraway, K. L., 3rd, and L. C. Cantley. 1994. A neu acquaintance for erbB3 and erbB4: a role for receptor heterodimerization in growth signaling. Cell. 78:5-8.
Carraway, K. L., 3rd, M. X. Sliwkowski, R. Akita, J. V. Platko, P. M. Guy, A. Nuijens, A. J. Diamonti, R. L. Vandlen, L. C. Cantley, and R. A. Cerione. 1994. The erbB3 gene product is a receptor for heregulin. J Biol Chem. 269:14303-14306.
Chan, B. T., and A. V. Lee. 2008. Insulin receptor substrates (IRSs) and breast tumorigenesis. Journal of mammary gland biology and neoplasia. 13:415-422.
Chan, S. D., D. M. Antoniucci, K. S. Fok, M. L. Alajoki, R. N. Harkins, S. A. Thompson, and H. G. Wada. 1995. Heregulin activation of extracellular acidification in mammary carcinoma cells is associated with expression of HER2 and HER3. J Biol Chem. 270:22608-22613.
Chi, H., M. R. Sarkisian, P. Rakic, and R. A. Flavell. 2005. Loss of mitogen-activated protein kinase kinase kinase 4 (MEKK4) results in enhanced apoptosis and defective neural tube development. Proc Natl Acad Sci USA. 102:3846-3851.
Darcy, K. M., D. Zangani, A. L. Wohlhueter, R. Y. Huang, M. M. Vaughan, J. A. Russell, and M. M. Ip. 2000. Changes in ErbB2 (her-2/neu), ErbB3, and ErbB4 during growth, differentiation, and apoptosis of normal rat mammary epithelial cells. The journal of histochemistry and cytochemistry: official journal of the Histochemistry Society. 48:63-80.
Daub, H., J. V. Olsen, M. Bairlein, F. Gnad, F. S. Oppermann, R. Korner, Z. Greff, G. Keri, 0. Stemmann, and M. Mann. 2008. Kinase-selective enrichment enables quantitative phosphoproteomics of the kinome across the cell cycle. Molecular cell. 31:438-448.
Derbyshire, Z. E., U. M. Halfter, R. L. Heimark, T. H. Sy, and R. R. Vaillancourt. 2005. Angiotensin II stimulated transcription of cyclooxygenase II is regulated by a novel kinase cascade involving Pyk2, MEKK4 and annexin II. Molecular and Cellular Biochemistry. 271:77-90.
Eccles, S. A. 2011. The epidermal growth factor receptor/Erb-B/HER family in normal and malignant breast biology. The International journal of developmental biology. 55:685-696.
Engel, R. H., and V. G. Kaklamani. 2007. HER2-positive breast cancer: current and future treatment strategies. Drugs. 67:1329-1341.
Esteva, F. J., and L. Pusztai. 2005. Optimizing outcomes in HER2-positive breast cancer: the molecular rationale. Oncology. 19:5-16.
Feldner, J. C., and B. H. Brandt. 2002. Cancer cell motility—on the road from c-erbB-2 receptor steered signaling to actin reorganization. Exp Cell Res. 272:93-108.
Fiddes, R. J., P. W. Janes, G. M. Sanderson, S. P. Sivertsen, R. L. Sutherland, and R. J. Daly. 1995. Heregulin (HRG)-induced mitogenic signaling and cytotoxic activity of a HRG/PE40 ligand toxin in human breast cancer cells. Cell Growth Differ. 6:1567-1577.
Fiddes, R. J., P. W. Janes, S. P. Sivertsen, R. L. Sutherland, E. A. Musgrove, and R. J. Daly. 1998. Inhibition of the MAP kinase cascade blocks heregulin-induced cell cycle progression in T-47D human breast cancer cells. Oncogene. 16:2803-2813.
Fuchs, I. B., I. Siemer, H. Buhler, A. Schmider, W. Henrich, W. Lichtenegger, G. Schaller, and S. Kuemmel. 2006. Epidermal growth factor receptor changes during breast cancer metastasis. Anticancer research. 26:4397-4401.
Garrett, J. T., M. G. Olivares, C. Rinehart, N. D. Granja-Ingram, V. Sanchez, A. Chakrabarty, B. Dave, R. S. Cook, W. Pao, E. McKinely, H. C. Manning, J. Chang, and C. L. Arteaga. 2011. Transcriptional and posttranslational up-regulation of HER3 (ErbB3) compensates for inhibition of the HER2 tyrosine kinase. Proc Natl Acad Sci USA. 108:5021-5026.
Gerwins, P., J. L. Blank, and G. L. Johnson. 1997. Cloning of a novel mitogen-activated protein kinase kinase kinase, MEKK4, that selectively regulates the c-Jun amino terminal kinase pathway. J Biol Chem. 272:8288-8295.
Halfter, U. M., Z. E. Derbyshire, and R. R. Vaillancourt. 2005. Interferon-gamma-dependent tyrosine phosphorylation of MEKK4 via Pyk2 is regulated by annexin II and SHP2 in keratinocytes. Biochemical journal. 388:17-28.
Hellyer, N.J., K. Cheng, and J. G. Koland. 1998. ErbB3 (HER3) interaction with the p85 regulatory subunit of phosphoinositide 3-kinase. The Biochemical journal. 333 (Pt 3):757-763.
Hernandez, L., T. Smirnova, D. Kedrin, J. Wyckoff, L. Zhu, E. R. Stanley, D. Cox, W. J. Muller, J. W. Pollard, N. Van Rooijen, and J. E. Segall. 2009. The EGF/CSF-1 paracrine invasion loop can be triggered by heregulin betal and CXCL12. Cancer Res. 69:3221-3227.
Hijazi, M. M., E. W. Thompson, C. Tang, P. Coopman, J. A. Toni, D. Yang, S. C. Mueller, and R. Lupu. 2000. Heregulin regulates the actin cytoskeleton and promotes invasive properties in breast cancer cell lines. International journal of oncology. 17:629-641.
Hoffman, I I., and S. Kaplan. 2002. The incidence of congenital heart disease. Journal of the American College of Cardiology. 39:1890-1900.
Holbro, T., R. R. Beerli, F. Maurer, M. Koziczak, C. F. Barbas, 3rd, and N. E. Hynes. 2003. The ErbB2/ErbB3 heterodimer functions as an oncogenic unit: ErbB2 requires ErbB3 to drive breast tumor cell proliferation. Proc Natl Acad Sci USA. 100:8933-8938.
Hynes, N. E., and H. A. Lane. 2005. ERBB receptors and cancer: the complexity of targeted inhibitors. Nature reviews. Cancer. 5:341-354.
Jones, R. B., A. Gordus, J. A. Krall, and G. MacBeath. 2006. A quantitative protein interaction network for the ErbB receptors using protein microarrays. Nature. 439:168-174.
Kanungo, J., I. Potapova, C. C. Malbon, and H. Wang. 2000. MEKK4 mediates differentiation in response to retinoic acid via activation of c-Jun N-terminal kinase in rat embryonal carcinoma P19 cells. J Biol Chem. 275:24032-24039.
Khazaie, K., V. Schirrmacher, and R. B. Lichtner. 1993. EGF receptor in neoplasia and metastasis. Cancer metastasis reviews. 12:255-274.
Kim, J. M., S. K. Min, H. Kim, H. K. Kang, S. Y. Jung, S. H. Lee, Y. Choi, S. Roh, D. Jeong, and B. M. Min. 2007. Vacuolar-type H+-ATPase-mediated acidosis promotes in vitro osteoclastogenesis via modulation of cell migration. International journal of molecular medicine. 19:393-400.
Kooijman, R., J. J. Lauf, A. C. Kappers, and G. T. Rijkers. 1995. Insulin-like growth factor induces phosphorylation of immunoreactive insulin receptor substrate and its association with phosphatidylinositol-3 kinase in human thymocytes. The Journal of experimental medicine. 182:593-597.
Kraus, M. H., W. Issing, T. Mild, N. C. Popescu, and S. A. Aaronson. 1989. Isolation and characterization of ERBB3, a third member of the ERBB/epidermal growth factor receptor family: evidence for overexpression in a subset of human mammary tumors. Proc Natl Acad Sci USA. 86:9193-9197.
Krueger, J. S., V. G. Keshamouni, N. Atanaskova, and K. B. Reddy. 2001. Temporal and quantitative regulation of mitogen-activated protein kinase (MAPK) modulates cell motility and invasion. Oncogene. 20:4209-4218.
Kurokawa, H., and C. L. Arteaga. 2003. ErbB (HER) receptors can abrogate antiestrogen action in human breast cancer by multiple signaling mechanisms. Clinical cancer research: an official journal of the American Association for Cancer Research. 9:511 S-515S.
Kurokawa, H., A. E. Lenferink, J. F. Simpson, P. I. Pisacane, M. X. Sliwkowski, J. T. Forbes, and C. L. Arteaga. 2000 Inhibition of HER2/neu (erbB-2) and mitogen-activated protein kinases enhances tamoxifen action against HER2-overexpressing, tamoxifen-resistant breast cancer cells. Cancer Res. 60:5887-5894.
Lange-Carter, C. A., C. M. Pleiman, A. M. Gardner, K. J. Blumer, and G. L. Johnson. 1993. A divergence in the MAP kinase regulatory network defined by MEK kinase and Raf. Science. 260:315-319.
Lemmon, M. A. 2003. The EGF receptor family as therapeutic targets in breast cancer. Breast Dis. 18:33-43.
Lyne, J. C., M. F. Melhem, G. G. Finley, D. Wen, N. Liu, D. H. Deng, and R. Salup. 1997. Tissue expression of neu differentiation factor/heregulin and its receptor complex in prostate cancer and its biologic effects on prostate cancer cells in vitro. The cancer journal from Scientific American. 3:21-30.
Maeda, T., M. Takekawa, and H. Saito. 1995. Activation of yeast PBS2 MAPKK by MAPKKKs or by binding of an SH3-containing osmosensor. Science. 269:554-558.
Menard, S., E. Tagliabue, M. Campiglio, and S. M. Pupa. 2000. Role of HER2 gene overexpression in breast carcinoma. Journal of cellular physiology. 182:150-162.
Mita, H., J. Tsutsui, M. Takekawa, E. A. Witten, and H. Saito. 2002. Regulation of MTK1/MEKK4 kinase activity by its N-terminal autoinhibitory domain and GADD45 binding. Mol Cell Biol. 22:4544-4555.
Miyake, Z., M. Takekawa, Q. Ge, and H. Saito. 2007. Activation of MTK1/MEKK4 by GADD45 through induced N-C dissociation and dimerization-mediated trans autophosphorylation of the MTK1 kinase domain. Mol Cell Biol. 27:2765-2776.
Muller, W. J., E. Sinn, P. K. Pattengale, R. Wallace, and P. Leder. 1988. Single-step induction of mammary adenocarcinoma in transgenic mice bearing the activated c-neu oncogene. Cell. 54:105-115.
Neve, R. M., T. Holbro, and N. E. Hynes. 2002. Distinct roles for phosphoinositide 3-kinase, mitogen-activated protein kinase and p38 MAPK in mediating cell cycle progression of breast cancer cells. Oncogene. 21:4567-4576.
Plowman, G. D., J. M. Green, J. M. Culouscou, G. W. Carlton, V. M. Rothwell, and S. Buckley. 1993. Heregulin induces tyrosine phosphorylation of HER4/p180erbB4. Nature. 366:473-475.
Rofstad, E. K., B. Mathiesen, K. Kindem, and K. Galappathi. 2006. Acidic extracellular pH promotes experimental metastasis of human melanoma cells in athymic nude mice. Cancer Res. 66:6699-6707.
Sarkisian, M. R., C. M. Bartley, H. Chi, F. Nakamura, K. Hashimoto-Torii, M. Torii, R. A. Flavell, and P. Rakic. 2006. MEKK4 signaling regulates filamin expression and neuronal migration. Neuron. 52:789-801.
Scaltriti, M., C. Verma, M. Guzman, J. Jimenez, J. L. Parra, K. Pedersen, D. J. Smith, S. Landolfi, S. Ramon y Cajal, J. Arribas, and J. Baselga. 2009. Lapatinib, a HER2 tyrosine kinase inhibitor, induces stabilization and accumulation of HER2 and potentiates trastuzumab-dependent cell cytotoxicity. Oncogene. 28:803-814.
Schmitz-Peiffer, C., and J. P. Whitehead. 2003. IRS-1 regulation in health and disease. IUBMB life. 55:367-374.
Sennoune, S. R., D. Luo, and R. Martinez-Zaguilan. 2004. Plasmalemmal vacuolar-type H+-ATPase in cancer biology. Cell biochemistry and biophysics. 40:185-206.
Shou, J., S. Massarweh, C. K. Osborne, A. E. Wakeling, S. Ali, H. Weiss, and R. Schiff. 2004. Mechanisms of tamoxifen resistance: increased estrogen receptor-HER2/neu cross-talk in ER/HER2-positive breast cancer. Journal of the National Cancer Institute. 96:926-935.
Smirnova, T., Z. N. Zhou, R. J. Flinn, J. Wyckoff, P. J. Boimel, M. Pozzuto, S. J. Coniglio, J. M. Backer, A. R. Bresnick, J. S. Condeelis, N. E. Hynes, and J. E. Segall. 2012. Phosphoinositide 3-kinase signaling is critical for ErbB3-driven breast cancer cell motility and metastasis. Oncogene. 31:706-715.
Soltoff, S. P., K. L. Carraway, 3rd, S. A. Prigent, W. G. Gullick, and L. C. Cantley. 1994. ErbB3 is involved in activation of phosphatidylinositol 3-kinase by epidermal growth factor. Mol Cell Biol. 14:3550-3558.
Stern, D. F. 2008. ERBB3/HER3 and ERBB2/HER2 duet in mammary development and breast cancer. Journal of mammary gland biology and neoplasia. 13:215-223.
Stern, H. M. 2012. Improving treatment of HER2-positive cancers: opportunities and challenges. Science translational medicine. 4:127rv122.
Stevens, M. V., P. Parker, R. R. Vaillancourt, and T. D. Camenisch. 2006. MEKK4 regulates developmental EMT in the embryonic heart. Dev Dyn. 235:2761-2770.
Stock, C., B. Gassner, C. R. Hauck, H. Arnold, S. Mally, J. A. Eble, P. Dieterich, and A. Schwab. 2005. Migration of human melanoma cells depends on extracellular pH and Na+/H+exchange. The Journal of physiology. 567:225-238.
Stock, C., and A. Schwab. 2009. Protons make tumor cells move like clockwork. Pflugers Archiv: European journal of physiology. 458:981-992.
Sun, B. K., J. H. Kim, H. N. Nguyen, S. Oh, S. Y. Kim, S. Choi, H. J. Choi, Y. J. Lee, and J. J. Song. 2011. MEKK1/MEKK4 are responsible for TRAIL-induced JNK/p38 phosphorylation. Oncology reports. 25:537-544.
Takekawa, M., F. Posas, and H. Saito. 1997. A human homolog of the yeast Ssk2/Ssk22 MAP kinase kinase kinases, MTK1, mediates stress-induced activation of the p38 and JNK pathways. Embo J. 16:4973-4982.
Takekawa, M., and H. Saito. 1998. A family of stress-inducible GADD45-like proteins mediate activation of the stress-responsive MTK1/MEKK4 MAPKKK. Cell. 95:521-530.
Teixido, C., E. Arguelaguet, B. Pons, M. Aracil, J. Jimeno, R. Somoza, R. Mares, Y. C. S. Ramon, and J. Hernandez-Losa. 2012. ErbB3 expression predicts sensitivity to elisidepsin treatment: in vitro synergism with cisplatin, paclitaxel and gemcitabine in lung, breast and colon cancer cell lines. International journal of oncology. 41:317-324.
Vaught, D. B., J. C. Stanford, C. Young, D. J. Hicks, F. Wheeler, C. Rinehart, V. Sanchez, J. Koland, W. J. Muller, C. L. Arteaga, and R. S. Cook. 2012. HER3 is required for HER2-induced preneoplastic changes to the breast epithelium and tumor formation. Cancer Res. 72:2672-2682.
Way, T. D., and J. K. Lin. 2005. Role of HER2/HER3 co-receptor in breast carcinogenesis. Future oncology. 1:841-849.
Wood, E. R., A. T. Truesdale, O. B. McDonald, D. Yuan, A. Hassell, S. H. Dickerson, B. Ellis, C. Pennisi, E. Horne, K. Lackey, K. J. Alligood, D. W. Rusnak, T. M. Gilmer, and L. Shewchuk. 2004. A unique structure for epidermal growth factor receptor bound to GW572016 (Lapatinib): relationships among protein conformation, inhibitor off-rate, and receptor activity in tumor cells. Cancer Res. 64:6652-6659.
Wu, W. S., J. R. Wu, and C. T. Hu. 2008. Signal cross talks for sustained MAPK activation and cell migration: the potential role of reactive oxygen species. Cancer metastasis reviews. 27:303-314.
Xue, C., F. Liang, R. Mahmood, M. Vuolo, J. Wyckoff, H. Qian, K. L. Tsai, M. Kim, J. Locker, Z. Y. Zhang, and J. E. Segall. 2006. ErbB3-dependent motility and intravasation in breast cancer metastasis. Cancer Res. 66:1418-1426.
Yamazaki, D., S. Kurisu, and T. Takenawa. 2005. Regulation of cancer cell motility through actin reorganization. Cancer science. 96:379-386.
Yang, C., E. A. Klein, R. K. Assoian, and M. G. Kazanietz. 2008. Heregulin betal promotes breast cancer cell proliferation through Rac/ERK-dependent induction of cyclin D1 and p21Cipl. The Biochemical journal. 410:167-175.
Yarden, R. I., A. H. Lauber, D. El-Ashry, and S. A. Chrysogelos. 1996. Bimodal regulation of epidermal growth factor receptor by estrogen in breast cancer cells. Endocrinology. 137:2739-2747.
Yarden, Y., and M. X. Sliwkowski. 2001. Untangling the ErbB signalling network. Nature Reviews Molecular Cell Biology. 2:127-137.
Yuzyuk, T., M. Foehr, and D. C. Amberg. 2002. The MEK kinase Ssk2p promotes actin cytoskeleton recovery after osmotic stress. Mol Biol Cell. 13:2869-2880.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating a subject suffering from a clinical condition associated with activation of HER3/ErbB3, said method comprising:
administering to said subject a therapeutically effective amount of a compound that inhibits interaction between actin and mitogen-activated protein three kinase (MTK1).

2. The method of claim 1, wherein said clinical condition associated with activation of HER3/ErbB3 comprises cancer, lung fibrosis, chronic obstructive pulmonary disease, or acidification of tumor metabolic microenvironment.

3. The method of claim 1, wherein said clinical condition associated with activation of HER3/ErbB3 comprises breast cancer, prostate cancer, ovarian cancer, neurological cancers, retinoblastomas, melanoma, gastrointestinal cancers, pancreatic cancer, colorectal cancers, head and neck cancers, or lung cancer.

4. The method of claim 1, wherein said MTK1-actin inhibitor comprises cytochalasin D.

5. The method of claim 1, wherein the compound inhibits interaction of actin with a region of MTK1 comprising amino acid sequence from 264 to 303.

6. A method for screening for an anticancer compound, said method comprising:
contacting a cell culture with a test compound under conditions sufficient to determine whether the test compound modulates interaction between actin and a mitogen-activated protein kinase kinase (MTK1); and
selecting the test compound that inhibits interaction between actin and MTK1 as an anticancer compound.

7. The method of claim 6, wherein said cell culture comprises HEK-293, T-47D, MCF7, or MDA-MB-231 cells.

8. A method for treating a patient suffering from a cancer, said method comprising administering to the patient a therapeutically effective amount of a composition comprising a molecule that inhibits interaction between actin and mitogen-activated protein three kinase (MTK1).

9. The method of claim 8, wherein said cancer comprises breast cancer, prostate cancer, ovarian cancer, neurological cancers, retinoblastomas, melanoma, gastrointestinal cancers, pancreatic cancer, colorectal cancers, head and neck cancers, or lung cancer.
